(19) United States
(12) Patent Application Publication
(10) Pub. No.: US 2005/0136033 A9
(43) Pub. Date: Jun. 23, 2005

(54) Method of treating allergen induced airway disease

(76) Inventors: Matthias Mack, Munchen (DE); Corey M. Hogaboam, Ann Arbor, MI (US)

Correspondence Address: FISH & RICHARDSON PC, 225 FRANKLIN ST, BOSTON, MA, 02110, US

(21) Appl. No.: 10/217,524

(22) Filed: Aug. 13, 2002

(51) Int. Cl. A61K 38/19

(52) U.S. Cl. 424/85.1

(57) ABSTRACT

The invention features methods of treating allergic asthma by administering to a subject a chimeric polypeptide that comprises a first polypeptide domain comprising at least one moiety that specifically binds to a chemokine receptor; and, a second polypeptide domain comprising at least one of (a)-(d): (a) a moiety that binds to a T cell surface polypeptide, (b) a moiety that binds to a dendritic cell surface polypeptide, (c) a moiety that binds to a cell toxin, or (d) a cell toxin.

### BACKGROUND OF THE INVENTION

Asthma is an inflammatory airway disease characterized by the presence of cells such as eosinophils, mast cells, basophils, and CD25+ T lymphocytes in the airway walls. Chemokines attract cells to the site of inflammation and cytokines activate them, resulting in inflammation and damage to the mucosa. When asthma becomes chronic, secondary changes occur, such as thickening of basement membrane and fibrosis.

In allergic asthma (also known as extrinsic asthma), the initiation event of airway inflammation is an immunological reaction to allergen. Continued exposure to allergen results in chronic inflammation. Allergic asthma affects about 3 million children (8 to 12 percent of all children) and 7 million adults in the United States at a cost estimated at $6.2 billion a year.

Regulated upon activation in normal T cells expressed and secreted (RANTES)/CCL5 has been implicated in the directed migration and/or activation of monocytes (2) and eosinophils (3) (4). Cellular sources of RANTES/CCL5 include alveolar macrophages (5), platelets (6), eosinophils (7), tissue-resident macrophages (8), human airway smooth-muscle cells (9), pulmonary-derived fibroblasts (10, 11), and airway epithelial cells (12-14). RANTES/CCL5 has been shown to bind and activate cells via CC chemokine receptor-1 (CCR1), CCR3, CCR4, and CCR5 (15). Blockade of CCR3 has been suggested as a strategy for asthma therapy (74).

### SUMMARY OF THE INVENTION

The invention is based, in part, on the inventors' discovery that administration of a chimeric polypeptide comprising a first polypeptide domain comprising at least one moiety that specifically binds to a chemokine receptor, e.g., CCR1, CCR4 or CCR5; and, a second polypeptide domain comprising at least one of (a)-(d): (a) a moiety that binds to a T cell surface polypeptide, (b) a moiety that binds to a dendritic cell surface polypeptide, (c) a moiety that binds to a cell toxin, or (d) a cell toxin, is effective to treat allergen-induced airway disease, e.g., allergen-induced asthma, e.g., chronic allergic asthma (e.g., childhood asthma), in a subject, e.g., a mammal, e.g., a human. Although the methods described herein are not bound by a particular mechanism or theory, it is believed that the chimeric polypeptide directly targets CCR4/CCR8-expressing cells or RANTES/CCL5-responsive cells (e.g., CCR5 expressing cells) that regulate the recruitment of CCR4/CCR8-expressing cells into allergen-challenged lungs. Thus, the chimeric polypeptide is believed to inhibit the initiation, e.g., the severity or rapidity of initiation, of chronic allergic airway disease.

Accordingly, in one aspect, the invention features a method of treating allergen-induced asthma, e.g., treating the initiation of allergen-induced asthma, e.g., the initiation of chronic allergic asthma, in a subject. The method includes administering to the subject (e.g., a mammal, e.g., a human) a chimeric polypeptide which includes a first polypeptide domain comprising at least one moiety that specifically binds to a chemokine receptor; and a second polypeptide domain comprising at least one of (a)-(d): (a) a moiety that binds to a T cell surface polypeptide, (b) a moiety that binds to a dendritic cell surface polypetide, (c) a moiety that binds to a cell toxin, or (d) a cell toxin.

Optionally, the method also includes identifying a subject who is at risk for, or has, allergic asthma, e.g., chronic allergic asthma. An ordinary skilled practitioner could identify such a subject by performing an art recognized diagnostic evaluation for allergic asthma. For example, a diagnosis of allergic asthma can be made by one or more of: administration of an allergen skin test; determination of IgE concentration in serum (e.g., IgE >300 ng/ml); and determination of specific IgE or IgG antibodies in serum (e.g., presence of IgG antibodies to Aspergillus antigen).

Optionally, the method also includes evaluating a symptom of asthma in the subject, e.g., airway hyperresponsiveness, coughing, wheezing, chest tightness, dyspnea, airway smooth muscle contraction, bronchial mucus secretion, inflammation, vasodilation, recruitment of inflammatory cells (e.g., neutrophils, monocytes, macrophages, lymphocytes, eosinophils), goblet cell hyperplasia, release of inflammatory mediators by mast cells or migrating inflammatory cells. The evaluation step can be performed before, during and/or after the administration step. The evaluation can be performed by a physician, other health care provider or by the subject. In some embodiments, the evaluation includes performing a bronchoalveolar lavage (BAL) or evaluating IgE levels in the subject. The evaluation can be performed one or more times, e.g., one or more times after administration, e.g., at least twice during a one week, one month, two month, three month, six month period after the administration, or longer. In a preferred embodiment, the method includes determining whether the administration of the chimeric polypeptide (or multiple administrations) reduced the severity or initiation of one or more symptoms of airway disease in the subject.

In preferred aspects of the methods described herein, administration of the chimeric polypeptide inhibits the initiation of chronic allergic airway disease, e.g., chronic allergic asthma. Thus, the chimeric polypeptide is preferably administered after exposure (or after suspected exposure or prior to impending exposure) to an allergen to which the subject is hypersensitive. For example, the chimeric polypeptide is administered at least once between 0 to 30 days, preferably between 0 to 20 days, more preferably between 0 to 15 days or between 0 to 7 days, after the subject has been exposed to the allergen to which the subject is hypersensitive.

In another aspect, the chimeric polypeptide is administered daily for a specified time, e.g., for a period of at least 3 days. For example, the chimeric polypeptide can be administered daily for at least one week, at least one month, at least 3 months, 6 months, a year or longer.

In some embodiments, the chimeric polypeptide is administered in a regimen that includes at least 1, preferably 2, more preferably 3, 4, 5, 10 or more cycles of daily treatment. A cycle includes: (a) a period during which the chimeric polypeptide is administered daily for 1-30 days, preferably for 2-20 days, more preferably for 2 to 15 days or for 2 to 7 days, followed by (b) a rest period of at least one day (preferably at least one week, 2 weeks, 3 weeks, or a month or more) in which the chimeric polypeptide is not administered. The number of days of administration and rest can be the same or different within a cycle. Likewise, two or more consecutive cycles can have the same or a different duration. For example, the chimeric polypeptide can be administered in a regimen that includes the following cycles in an exemplary 10 week period: four weeks of daily administration followed by one week of rest (cycle 1), two weeks of administration followed by one week of rest (cycle 2), one week of administration followed by one week of rest (cycle 3). In another example, the chimeric polypeptide can be administered in a 10 week regimen that includes 35 cycles of: one day of administration followed by one day of rest. Other combinations of such cycle therapy can be routinely established by a skilled practitioner according to the needs of a particular patient.

In a preferred embodiment, goblet cell hyperplasia is reduced in the subject after administration, e.g., between 1 and 20 days, more preferably between 1 and 15 days or between 1 and 7 days after administration.

In a preferred embodiment, the chemokine receptor is a chemokine receptor 5 (CCR5), such as a human chemokine receptor 5 (CCR5).

In another preferred embodiment, the chemokine receptor is a CCR4, e.g., human CCR4. For example, in such embodiments, the chimeric polypeptide directly targets CCR4/CCR8-expressing cells. In some embodiments, the expression of CCR4 and/or CCR8 ligands, e.g., MDC/CCL22 and TARC/CCL17 (65, 66), are reduced in the lung.

In other embodiments, the chemokine receptor is CCR1 (see, e.g., Hesselgesser (1998) J. Biol. Chem. 273:15687-15692), CCR3 (see, e.g., Dairaghi (1997) J. Biol. Chem. 272:28206-28209), CCR4 (see, e.g., Imai (1997) J. Biol. Chem. 272:15036-15042), CCR8 (see, e.g., Roos (1997) J. Biol. Chem. 272:17251-17254), CXCR4 (see, e.g., Vila-Coro (1999) FASEB J. 13:1699-1710), CXCR5 (see, e.g., Legler (1998) J. Exp. Med. 187:655-660), CXCR6 (see, e.g., Luttichau (2001) Eur. J. Immunol. 31:1217-1220), CCR2 (see, e.g., Monteclaro (1997) J. Biol. Chem. 272:23186-23190), CCR5 (see, e.g., Ganju (2000) J. Biol. Chem. 275:17263-17268), CCR6 (see, e.g., Baba (1997) J. Biol. Chem. 272:14893-14898), CCR7 (see, e.g., Kim (1999) Cell Immunol. 193:226-235), CCR9 (see, e.g., Norment (2000) J. Immunol. 164:639-648), CCR10 (see, e.g., Bonini (1997) DNA Cell Biol. 16:1249-1256), XCR1 (GPR5) (see, e.g., Shan (2000) Biochem. Biophys. Res. Commun. 268:938-941), or CX3CR1 (see, e.g., Combadiere (1998) Biochem. Biophys. Res. Commun. 253:728-732); see Table I, which includes the corresponding chemokine ligands. Chemokines and/or chemokine ligands binding to the chemokine receptors are well known in the art; examples of receptors and corresponding ligands are shown, inter alia, in Table I. Furthermore, chemokines and their corresponding receptors are disclosed in, e.g., U.S. Pat. Nos. 6,174,995; 6,172,061; 6,166,015; Murphy (2000) Pharm. Reviews 52:145-176. The moiety that specifically binds to a chemokine receptor can be a polypeptide or any other molecule, such as a synthetic chemical, that specifically binds to a chemokine receptor.

In a preferred embodiment, the T cell surface polypeptide is a CD3 polypeptide.

In a preferred embodiment, the dendritic cell surface polypeptide is an Eo1 chemokine receptor (73).

The chimeric polypeptide can be a recombinant fusion protein comprising a first polypeptide domain comprising at least one moiety that specifically binds to a chemokine receptor; and, a second polypeptide domain comprising at least one of (a)-(d): (a) a moiety that binds to a T cell surface polypeptide, (b) a moiety that binds to a dendritic cell, (c) a moiety that binds to a cell toxin, or (d) a cell toxin.

In one embodiment, the moiety that specifically binds to the chemokine receptor 5 (CCR5) can comprise a RANTES (“regulated on activation normal T cell expressed and secreted”) polypeptide, or a fragment thereof capable of binding to a CCR5 or CCR4 receptor. More specifically, the moiety that specifically binds to the CCR5 chemokine receptor can comprise a MIP-1α, or a fragment thereof capable of binding to a CCR5 receptor. Alternatively, the moiety that specifically binds to the CCR5 chemokine receptor can comprise a MIP-1β, a MCP-2, or a MCP-3 or a fragment thereof, capable of binding to the CCR5 receptor. In other embodiments, the moiety that specifically binds to the chemokine receptor comprises an IP-10 (CXCL-10) (see, e.g., Agostini (2001) Am. J. Pathol. 158:1703-1711; Flier (1999) J. Invest. Dermatol. 113:574-578), or a Mig (CXCL9) (see, e.g., Farber (1997) J. Leukoc. Biol. 61:246-257), or an I-TAC (CXCL11) chemokine ligand (see, e.g., Gasperini (1999) J. Immunol. 162:4928-4937), or a fragment thereof (see Table I), capable of binding to the CXCR3 chemokine receptor.

In another embodiment, the moiety that specifically binds to the chemokine receptor is an antigen binding domain of an antibody. For example, the chimeric polypeptide can be a bispecific antibody comprising a first antigen binding domain that specifically binds to a chemokine receptor 5 (CCR5); and, a second antigen binding domain that specifically binds to a T cell surface polypeptide, a cell toxin, or a third antigen binding domain that specifically binds to or is linked to a T cell surface polypeptide or a cell toxin. The bispecific antibody is not limited to two binding domains. The T cell surface polypeptide can comprise a CD3 antigen.

In one embodiment, the bispecific antibody is a single chain antibody construct. The single chain antibody construct can comprise a VL and a VH domain capable of specifically binding the chemokine receptor and a VH and a VL domain capable of specifically binding a T cell surface polypeptide. In one exemplary embodiment, the antigen binding domain that specifically binds to a chemokine receptor can comprise a murine anti-human CCR5 antibody MC-1 (See Briühl et al., 2001, J Immunol 166(4):2420-26 and co-pending U.S. patent application Ser. No. 09/948,004, filed Sep. 5, 2001, owned by the same assignee as the present application).

In another embodiment, the second antigen-binding domain of the bispecific antibody specifically binds to a cell toxin, or, the second antigen binding domain specifically binds to another domain (e.g., an antibody) that can specifically bind a toxin (or a cell surface protein). Alternatively, the antibody is covalently bound (directly or indirectly) to a cell toxin. The antibody can be bound to a second antibody that binds to a CD3 antigen or a cell toxin.

In some embodiments, the chimeric polypeptide is co-administered with a second agent effective to treat asthma in the subject, e.g., the chimeric polypeptide can be co-administered with a corticosteroid, bronchodilator, leukotriene modifier, anti-inflammatory agent, or therapeutic antibody or funational fragment thereof, e.g., anti-IgE, anti-IL-9, anti-IL-3, anti-IL4, anti-IL-5, anti-VLA, or anti-migration inhibitory factor (MIF) antibody or functional fragment thereof. “Co-administered” or “administered in combination” means that the chimeric polypeptide is administered to a subject at the same time or within an interval, e.g., a week, such that the effects of the substances on the patient overlap.

In some embodiments, the chimeric polypeptide comprises a cell toxin, or a fragment or domain thereof that remains toxic to cells. The cell toxin can comprise a Pseudomonas exotoxin, or toxic fragment thereof. The Pseudomonas exotoxin can comprise a PE38 exotoxin, a PE40 exotoxin or a PE37 exotoxin. The cell toxin can also include a diptheria toxin. The cell toxin can be non-covalently or covalently, directly or indirectly, attached or associated with the chimeric polypeptide. In one embodiment, the toxin is cross-linked to the chimeric polypeptide. Alternatively, the toxin can comprise a recombinant fusion protein, as all or a portion of the chimeric polypeptide can comprise a recombinant protein, e.g., it can be a fusion protein. In one aspect, the moiety that specifically binds to a chemokine receptor comprises an antigen binding domain derived from an antibody that specifically binds to the chemokine receptor. The moiety that specifically binds to a T cell surface polypeptide can comprise an antigen binding domain derived from an antibody that specifically binds to the T cell surface polypeptide. The moiety that specifically binds to a cell toxin can comprise an antigen binding domain derived from an antibody that specifically binds to the cell toxin.

In an exemplary embodiment of the methods described herein, the chimeric polypeptide is a chemokine construct. For example, the chimeric polypeptide comprises a binding site for a CCR5 or CCR4, e.g., the polypeptide comprises RANTES (“regulated on activation normal T cell expressed and secreted”), e.g., human RANTES, and a toxin, a PE38 exotoxin or fragment thereof. The nucleotide and amino acid sequence of human RANTES is known in the art and can be found in (1) or at GenBank Accession No. M21121 or AF043341.

The chimeric polypeptide can be administered by administering the chimeric polypeptide directly to the subject, e.g., orally, intravenously (i.v.), subcutaneously, intranasally, intrabronchially (e.g., by inhalation); by administering to the subject a nucleic acid encoding the chimeric polypeptide described herein, e.g., by administering a vector comprising a nucleic acid encoding the chimeric polypeptide described herein; or by administering to the subject a cell, e.g., a mammalian cell, e.g., a human cell (e.g., a human fibroblast), that has been transfected with a nucleic acid encoding the chimeric polypeptide. The transfected cell can be implanted in the subject, wherein the cell can produce the chimeric polypeptide in situ.

In another aspect, the invention features a kit that includes a pharmaceutical composition that includes a chimeric polypeptide (e.g., a bispecific antibody) described herein; and instructions to administer the pharmaceutical composition to treat allergen induced airway disease, e.g., allergen-induced asthma, e.g., chronic allergic asthma or childhood asthma, in a subject. The chimeric polypeptide can include a covalently bound cell toxin. The cell toxin can be a truncated sequence, e.g., a domain of a full length toxin, that remains toxic to the cell, e.g., a Pseudomonas exotoxin A (PE38).

The kit can further include specific instructions for administration of the pharmaceutical composition, e.g., instructions for intranasal or intrabronchial administration (e.g., by inhalation), subcutaneous, intravenous or oral administration. The kit can further include a second agent effective to treat a symptom of asthma, e.g., a corticosteroid, bronchodilator, leukotriene inhibitor, anti-inflammatory agent or therapeutic antibody or functional fragment thereof. In some embodiments, the kit can include, instead of or in addition to the chimeric polypeptide, a nucleic acid or vector that includes a nucleotide sequence encoding a chimeric polypeptide described herein, or a cell transfected with a nucleic acid or vector that includes a nucleotide sequence encoding a chimeric polypeptide described herein.

The invention also provides the use of a chimeric polypeptide, as described herein, that comprises a first polypeptide domain comprising at least one moiety that specifically binds to a chemokine receptor; and, a second polypeptide domain comprising at least one of (a)-(d): (a) a moiety that binds to a T cell surface polypeptide, (b) a moiety that binds to a dendritic cell surface polypeptide, (c) a moiety that binds to a cell toxin, or (d) a cell toxin, in the manufacture or use of a medicament for the treatment of allergen induced airway disease, e.g., allergen-induced asthma, e.g., chronic allergen induced asthma, e.g., chronic childhood asthma.

In another aspect, the invention also features a method of providing a treatment for an allergen-induced airway disease, e.g., allergic asthma, e.g., chronic allergic asthma, comprising the following steps: (a) providing a pharmaceutical composition comprising a chimeric polypeptide (e.g., a chemokine construct, e.g., RANTES-PE38) or a nucleic acid of the invention (e.g., a vector of the invention), and (b) providing instructions to use the pharmaceutical composition to treat the allergen-induced airway disease, e.g., allergic asthma, e.g., chronic allergic asthma.

As used herein, an “allergen” is a substance that causes airway hyperreactivity in a subject following a prior exposure and sensitization. Allergens can include any of: dust mites, cockroaches, pollen, mold, fungus, smog, pet dander or pet hair. In many instances, a specific subject has a particular sensitivity to a specific allergen.

In accordance with the present invention, the term “antibody and/or chemokine construct” not only comprises the molecules and multifunctional constructs and compounds as described herein, but also comprises functional fragments thereof. Functional fragments of the constructs are meant to be fragments which are capable of binding to/interacting with a desired molecule on a target cell, e.g., a chemokine receptor on a target cell, thus providing means for depleting, lysing and/or destroying the target cell.

### DESCRIPTION OF THE DRAWINGS

FIG. 1 is a graph showing whole lung levels of RANTES/CCL5 in A. fumigatus-sensitized CBA/J mice prior to and at days 3, 7, 8, 14, and 30 after a live A. fumigatus conidia challenge. RANTES/CCL5 levels were measured using a specific ELISA as described herein below. Data are expressed as mean ±SE; n=5/group/time point.

FIG. 2 is a graph showing whole lung levels of RANTES/CCL5 in A. fumigatus-sensitized CBA/J mice at days 3, 7, and 14 after a live A. fumigatus conidia challenge combined with 2.0×10⁸ plaque forming units (PFU) of either AdLacZ or AdRANTES/CCL5. RANTES/CCL5 was measured in both groups using a specific ELISA as described herein below. Data are expressed as mean ±SE; n=4-5/group/time point.

FIG. 3 is a graph showing airway hyperresponsiveness in A. fumigatus-sensitized CBA/J mice at days 3, 7, 14 after a live A. fumigatus conidia challenge combined with 2.0×10⁸ plaque forming units (PFU) of either AdLacZ or AdRANTES/CCL5. Peak increases in airway resistance or hyperresponsiveness (units=cm H₂O/ml/s) were determined at each time point after the intravenous injection of methacholine. Data are expressed as mean ±SE; n=4-5/group/time point.

FIG. 4 is a graph showing leukocyte counts in bronchoalveolar lavage (BAL) samples from A. fumigatus-sensitized CBA/J mice at days 3, 7, and 14 after a live A. fumigatus conidia challenge combined with 2.0×10⁸ plaque forming units (PFU) of either AdLacZ or AdRANTES/CCL5. BAL cells were dispersed onto microscope slides using a cytospin, and eosinophils, neutrophils, T lymphocytes and macrophages were differentially stained with Wright-Giesma stain. A minimum of 15 high-powered fields or 300 cells was examined in each cytospin. A total of 1×10⁵ BAL cells were cytospun onto each slide to compensate for differences in cell retrieval from each mouse. Values are expressed as mean ±SE. n=4-5/group/time point.

FIG. 5 shows representative photomicrographs of hematoxylin and eosin (H/E)-stained whole lung sections from in A. fumigatus-sensitized CBA/J mice at days 3 (A&B), 7 (C&D) after a live A. fumigatus conidia challenge combined with 2.0×10⁸ plaque forming units (PFU) of either AdLacZ or AdRANTES. Peribronchial inflammation was less prominent in lung sections from allergic mice that received Ad70-3 and conidia (A and C, respectively) compared with mice that received AdRANTES and conidia (B, and D, respectively). Original photomicrograph magnification was 200× for all panels.

FIG. 6 is graph showing whole lung levels of eotaxin/CCL11(A) and macrophage-derived chemokine (MDC/CCL22)(B) in A. fumigatus-sensitized CBA/J mice at days 3, 7, and 14 after a live A. fumigatus conidia challenge combined with 2.0×10⁸ plaque forming units (PFU) of either AdLacZ or AdRANTES/CCL5. MDC/CCL22 and eotaxin/CCL11 was measured in both groups using a specific ELISA as described herein below. Data are expressed as mean ±SE; n=4-5/group/time point.

FIG. 7 is graph showing airway hyperresponsiveness in A. fumigatus-sensitized CBA/J mice at day 15 (A) or 30 (B) after an intrapulmonary challenge with live A. fumigatus conidia. In the study summarized in Panel A, each mouse received 1 μg of RANTES-PE38 in 20 μl of saline or 20 μl of saline alone by intranasal delivery approximately 2 h prior to the conidia challenge. Subsequent to this first treatment and the conidia challenge, each mouse received RANTES-PE38 or saline every 48-h for 15 days. In the study summarized in Panel B, each mouse received 1 μg of RANTES-PE38 in 20 μl of saline or 20 μl of saline alone by intranasal delivery at day 15 after the conidia challenge. Subsequently, each mouse received RANTESPE38 or saline every 48-h up to day 30 after conidia. Peak increases in airway resistance or hyperresponsiveness (units=cm H₂O/ml/s) were determined at each time point after the intravenous injection of methacholine. Values are expressed as mean ±SE; n=5-10/group/time point.

FIG. 8 shows representative photomicrographs of hematoxylin and eosin (H/E)-stained whole lung sections at days 15 (A&B) and 30 (C&D) after an intrapulmonary challenge with live A. fumigatus conidia. Panels A & B: Mice received 1 μg of RANTES-PE38 in 20 μl of saline or 20 μl of saline alone by intranasal delivery approximately 2 h prior to the conidia challenge. Subsequent to this first treatment and the conidia challenge, each mouse received RANTESPE38 or saline every 48-h for 15 days. Panels C & D: each mouse received 1 μg of RANTESPE38 in 20 μl of saline or 20 μl of saline alone by intranasal delivery at day 15 after the conidia challenge. Subsequently, each mouse received RANTES-PE38 or saline every 48-h up to day 30 after conidia. Peribronchial inflammation was markedly greater in lung sections from saline-treated mice at days 15 (A) and 30 (C) compared with RANTES-PE38-treated mice at the same times after conidia (B&D, respectively). Original photomicrograph magnification was 200× for all panels.

FIG. 9 is a graph showing serum levels of IgE (A), IgG1 (B), and IgG2a (C) in A. fumigatus-sensitized mice at days 15 and 30 after a live A. fumigatus conidia challenge. In the day 15 group, mice received 1 μg of RANTES-PE38 in 20 μl of saline or 20 μl of saline alone by intranasal delivery approximately 2 h prior to the conidia challenge. Subsequent to this first treatment and the conidia challenge, each mouse received RANTES-PE38 or saline every 48-h for 15 days. In the day 30 group, each mouse received 1 μg of RANTES-PE38 in 20 μl of saline or 20 μl of saline alone by intranasal delivery at day 15 after the conidia challenge. Subsequently, each mouse received RANTES-PE38 or saline every 48-h up to day 30 after conidia. Serum Ig levels were measured using a specific ELISA as described herein below. Data are expressed as mean ±SE; n=5-10/group/time point.

FIG. 10 shows a GEArray Q series SuperArray analysis of chemokines and chemokine receptors in whole lung samples from A. fumigatus-sensitized CBA/J mice at days 15 (top panel) and 30 (bottom panel) after a live A. fumigatus conidia challenge. Top panel: mice received 1 μg of RANTES-PE38 in 20 μl of saline or 20 μl of saline alone by intranasal delivery approximately 2 h prior to the conidia challenge. Subsequent to this first treatment and the conidia challenge, each mouse received RANTES-PE38 or saline every 48-h for 15 days. Bottom panel: each mouse received 1 μg of RANTES-PE38 in 20 μl of saline or 20 μl of saline alone by intranasal delivery at day 15 after the conidia challenge. Subsequently, each mouse received RANTESPE38 or saline every 48-h up to day 30 after conidia. SuperArray analysis was used as described in the Materials and Methods section. Data are expressed as mean ±SE; n=3/group/time point.

FIG. 11 is a graph showing whole lung levels of macrophage derived chemokine (MDC/CCL22) (A) and TARC/CCL17 (B) in A. fumigatus-sensitized CBA/J mice at days 15 and 30 after a live A. fumigatus conidia challenge. In the day 15 group, mice received 1 μg of RANTES-PE38 in 20 μl of saline or 20 μl of saline alone by intranasal delivery approximately 2 h prior to the conidia challenge. Subsequent to this first treatment and the conidia challenge, each mouse received RANTES-PE38 or saline every 48-h for 15 days. In the day 30 group, each mouse received 1 μg of RANTES-PE38 in 20 μl of saline or 20 μl of saline alone by intranasal delivery at day 15 after the conidia challenge. Subsequently, each mouse received RANTESPE38 or saline every 48-h up to day 30 after conidia. Chemokines were measured in both groups using specific ELISAs as described herein below. Data are expressed as mean ±SE; n=5-10/group/time point.

FIG. 12 shows representative photomicrographs of Periodic acid Schiff (PAS)-stained whole lung sections at days 15 (A&B) and 30 (C&D) after an intrapulmonary challenge with live A. fumigatus conidia. Mice received 1 μg of RANTES-PE38 in 20 μl of saline or 20 μl of saline alone by intranasal delivery approximately 2 h prior to the conidia challenge. Subsequent to this first treatment and the conidia challenge, each mouse received RANTES-PE38 or saline every 48-h for 15 days. In the day 30 group, each mouse received 1 μg of RANTES-PE38 in 20 μl of saline or 20 μl of saline alone by intranasal delivery at day 15 after the conidia challenge. Subsequently, each mouse received RANTES-PE38 or saline every 48-h up to day 30 after conidia. Goblet cells (magenta-colored cells) were prominent in lung sections from saline-treated mice at days 15 (A) and 30 (C) whereas goblet cells were not apparent in RANTES-PE38-treated mice at day 15 after conidia (B). Goblet cells were detected in whole lung sections from RANTES-PE38-treated mice at day 30 after conidia (D). Original photomicrograph magnification was 200× for all panels.

FIG. 13 is a graph showing whole lung levels of neutrophil myeloperoxidase (MPO) (A) and monocyte chemoattractant protein-1 (MCP-1/CCL2)(B) in A. fumigatus-sensitized CBA/J mice at days 15 and 30 after a live A. fumigatus conidia challenge. In the day 15 group, mice received 1 μg of RANTES-PE38 in 20 μl of saline or 20 μl of saline alone by intranasal delivery approximately 2 h prior to the conidia challenge. Subsequent to this first treatment and the conidia challenge, each mouse received RANTES-PE38 or saline every 48-h for 15 days. In the day 30 group, each mouse received 1 μg of RANTES-PE38 in 20 μl of saline or 20 μl of saline alone by intranasal delivery at day 15 after the conidia challenge. Subsequently, each mouse received RANTES-PE38 or saline every 48-h up to day 30 after conidia. MPO and MCP1/CCL2 were measured in both groups using specific ELISAs as described in the Materials and Methods section. Data are expressed as mean ±SE; n=5-10/group/time point.

FIG. 14 is a schematic of the chemokine-toxin RANTES-PE38 chimeric polypeptide. The chemokine RANTES is fused to the N-terminus of a truncated version of the Pseudomonas exotoxin A (PE38). While the truncated toxin alone is unable to bind to eukaryotic cells, the fusion protein binds with the RANTES moiety to CCR5 and becomes internalized into the cell and, once intracellular, the toxin can inhibit protein synthesis and induce cell death.

FIG. 15(A-C) shows the nucleotide and amino acid sequence of a RANTES-PE38 chimeric polypeptide.

### DETAILED DESCRIPTION

The invention is based, in part, on the inventors' discovery that RANTES/CCL5 is directly involved in initiating an allergic pulmonary response and that the targeting of RANTES/CCL5-responsive cells (e.g., CCR5 expressing cells and/or CCR4/CCR8 expressing cells) offers a therapeutic approach to inhibit the initiation, e.g., the severity or rapidity of initiation, of allergic airway disease, e.g., chronic allergic airway disease, e.g., chronic allergic asthma.

It was found that allergic airway disease is transiently expressed in CCR5-deficient (−/−) mice and the temporal appearance of disease induced by an allergen in these mice was dependent on RANTES/CCL5 (29). As described herein, it has also been found that RANTES/CCL5 initiates the allergic pulmonary responses elicited by allergen (e.g., Aspergillus conidia) and that allergic asthma, e.g., initiation of chronic allergic asthma, can be treated, e.g., in a sensitized subject, by targeting of RANTES/CCL5-responsive cells with a chimeric polypeptide comprising a first polypeptide domain comprising at least one moiety that specifically binds to a chemokine receptor, e.g., CCR1, CCR4 or CCR5; and, a second polypeptide domain comprising at least one of (a)-(d): (a) a moiety that binds to a T cell surface polypeptide, (b) a moiety that binds to a dendritic cell surface polypeptide, (c) a moiety that binds to a cell toxin, or (d) a cell toxin.

The examples described herein below show that adenovirus-mediated over-expression of RANTES/CCL5 significantly enhanced the severity of allergic disease in A. fumigatus-sensitized mice intratracheally challenged with conidia, and the increased severity of disease was associated with increases in eosinophil and lymphocyte numbers in the lungs. Conversely, the intranasal delivery of RANTES-PE38 from the time of the conidia challenge up to day 15 significantly prevented the development of all airway features of chronic Aspergillus-induced allergic airway disease. The delayed delivery of RANTES-PE38 from days 15 to 30 after the conidia challenge had a significant inhibitory effect on airway inflammation and hyperreactivity but this treatment protocol did not reverse established allergic airway remodeling characterized by goblet cell hyperplasia. Thus, these experiments suggest that RANTES/CCL5 is a major initiator of chronic allergic airway disease due to Aspergillus, and as such is an appropriate target in the early treatment of this disease.

Other experiments described in the examples herein below demonstrated that the specific targeting of RANTES/CCL5-responsive cells with intranasal RANTES-PE38 treatments significantly attenuated the allergic airway disease induced by the intratracheal introduction of conidia into A. fumigatus-sensitized mice. RANTES-PE38 is a chimeric protein comprised of human RANTES and Pseudomonas exotoxin and it has been shown to efficiently deplete RANTES/CCL5-responsive, CCR5-positive cells (30). RANTES-PE38 can rapidly target cells expressing chemokine receptors such as CCR1, CCR3, CCR4 and CCR5. In the present examples, it is demonstrated that intranasal delivery of RANTES-PE38 has a major therapeutic effect in mice with allergic airway disease, e.g., chronic airway disease. In particular, RANTESPE38 had a therapeutic effect when A. fumigatus-induced allergic airway disease was established.

Allergic Asthma

Asthma is a variable and reversible obstruction of the respiratory tract due to hyperreactivity and inflammation of the respiratory tract. Two types are recognized, extrinsic and intrinsic asthma. Allergic (extrinsic) asthma has an allergic trigger and is caused by an IgE mediated response to an inhaled allergen, resulting in airway mast cell degranulation, mediator release, and the beginning of the inflammatory cascade. This type of asthma typically appears before the fourth decade of life, and is the most common type of childhood asthma. Intrinsic asthma generally develops later in life and virtually nothing is known of its root cause. Intrinsic bronchial hyperactivity can be triggered by infection, exercise, or drugs, e.g., aspirin. Intrinsic asthma carries a worse prognosis than extrinsic asthma.

Asthma in general is most often diagnosed from episodes of wheezing, coughing, chest tightness, and shortness of breath. Diagnostic pulmonary function tests include peak expiratory flow rate (PEFR) using a peak flow meter, or spirometry, which indicates forced vital capacity (FVC) and forced expiratory volume in 1 second (FEV1). Allergic asthma can be diagnosed from the presence of a temporal relationship between exposure to a putative aeroallergen and the development of airway symptoms (either early phase, late phase, or both), and from demonstrable skin test reactivity to the inhaled allergen. In many cases the symptoms of allergic asthma will be accompanied by symptoms of allergic rhinitis (rhinorrhea, sneezing, ocular burning). Allergic asthma can also de diagnosed from the presence of increased IgE levels (e.g., IgE >300 ng/ml in serum), or atopy. Intrinsic asthma can be diagnosed from the absence of atopy and negative results in skin tests to common allergens.

A number of therapeutic agents are useful in the managements and treatment of asthma. These include, but are not limited to, bronchodilators, e.g., anticholinergic bronchodilators to relax the airway (e.g., ipratropium bromide, albuterol/ipratropium bromide); beta agonists to relax airway muscles (e.g., epinephrine, metaproterenol, terbutaline, isoetharinemesylate, isoetharine, isuprel, pirbuterol, albuterol, salmeterol, bitolterol); oral or inhaled corticosteroids to reduce inflammation (e.g., hydrocortisone, cortisone, dexamethasone, prednisolone, prednisone, methylprednisolone, flunisolide, triamcinolone, beclomethosone, dexamethasone, fluticasone, budesonide); leukotriene modifiers to prevent the airways from swelling and blocking airflow and decrease mucus production (e.g., zafirlukast, montelukast sodium, zileuton); and theophylline, which helps, inter alia, to open the airways and reduce release of phlegm. Anti-asthma agents also include therapeutic antibodies (or functional fragments thereof), including, but not limited to, anti-IgE, anti-IL-9, anti-IL-3, anti-IL-4, anti-IL-5, anti-VLA, and anti-migration inhibitory factor (MIF). The chimeric polypeptides described herein can be administered in combination with one or more of the aforementioned agents to treat allergic asthma.

Chimeric Polypeptides

In one aspect of the methods of the invention, the chimeric polypeptide administered is an antibody construct, e.g., a bispecific antibody that binds to a desired chemokine receptor, such as a CCR5 or CCR4, as a first antigen and an antigen of an effector cell (e.g., CD3 or Eo1) as a second antigen. The CD3 antigen can be on the surface of a T-cell, such as a cytotoxic T-cell. CD3 is an antigen that is expressed on T cells and may be part of a multimolecular (T-) cell receptor complex.

Bispecific antibodies may be constructed by hybrid-hybridoma techniques, by covalently linking specific antibodies or by other approaches, like the diabody approach (Kipriyanow, Int. J. Cancer 77 (1998), 763-773). In one aspect of the invention, the bispecific antibody is a single chain antibody construct.

As is known, Fv, the minimum antibody fragment which contains a complete antigen recognition and binding site, consists of a dimer of one heavy and one light chain variable domain (V_{H} and V_{L}) in non-covalent association. In this configuration that corresponds to the one found in native antibodies the three complementarity determining regions (CDRs) of each variable domain interact to define an antigen binding site on the surface of the V_{H}-V_{L} dimer. Collectively, the six CDRs confer antigen binding specificity to the antibody. Frameworks (FRs) flanking the CDRs have a tertiary structure that is essentially conserved in native immunoglobulins of species as diverse as human and mouse. These FRs serve to hold the CDRs in their appropriate orientation. The constant domains are not required for binding function, but may aid in stabilizing V_{H}-V_{L} interaction. Even a single variable domain (or half of an Fv comprising only three CDRs specific for an antigen) has the ability to recognize and bind antigen, although usually at a lower affinity than an entire binding site (Painter, Biochem. 11 (1972), 1327-1337). Hence, said domain of the binding site of the antibody construct as defined and described in the present invention can be a pair of V_{H}-V_{L}, V_{H}-V_{H} or V_{L}-V_{L} domains of different immunoglobulins. The order of V_{H} and V_{L} domains within the polypeptide chain is not decisive for the present invention, the order of domains given hereinabove may be reversed usually without any loss of function. It is important, however, that the V_{H} and V_{L} domains are arranged so that the antigen binding site can properly fold.

Different parts of the antibodies/immunoglobulins can be joined by means of conventional methods or constructed as a contiguous protein by means of recombinant DNA techniques, e.g. in such a way that a nucleic acid molecule coding for a chimeric or humanized antibody chain is expressed in order to construct a contiguous protein (e.g., see Mack (1995) Proc. Natl. Acad. Sci. USA 92:7021-7025).

In one aspect, a single-chain antibody with the following Fv fragments is used: sc-Fv fragment of a monoclonal antibody against the chemokine receptor, such as, e.g., CCR5 or CCR4, and an sc-Fv fragment of a monoclonal antibody against a T cell surface polypeptide, e.g., a CD3 or a dendritic cell surface polypeptide, e.g., Eo1. In this case, both the Fv fragment directed against the chemokine receptor and the Fv fragment against CD3 or Eo1 may be located in N-terminal position. The Fv fragment against CCR5 may be in N-terminal position. The order of the VL and VH antibody domains can be variable in both constructs. In one aspect, the order of the Fv fragment against CCR5 is VL-VH and the one of the Fv fragment against CD3 is VH-VL. The linkers between the variable domains as well between the two Fv fragments may consist of peptide linkers, preferably of a hydrophilic flexible glycine- and serine-containing linker of 1 to 25 amino acids. An additional epitope tag, e.g., a histidine chain of, e.g., 6× His, in C- or N-terminal position, can be used to simplify purification and detection.

Compared to conventional bispecific antibodies, bispecific single-chain antibodies have the advantage that they consist of only one protein chain and thus their composition is exactly defined. They have a low molecular weight of normally <60 kD and can be produced easily and on a large scale in suitable cell lines, e.g. in CHO cells, using recombinant techniques. The most essential advantage, however, is that they have no constant antibody domains and thus only activate T-lymphocytes to lysis when these are bound to their target cells, i.e. to the chemokine-receptor expressing cells. Therefore, single-chain antibodies are often superior to conventional bispecific antibodies as their clinical use entails fewer or less severe side effects.

Therefore, in one aspect, the single chain antibody construct comprises V_{L} and V_{H} domains of a antibody specific for a chemokine receptor, such as a human CCR5, and V_{H} and V_{L} domains of an antibody specific for a T cell surface polypeptide, e.g., a CD3 antigen. One exemplary antibody specific for the human CCR5 is the murine anti-human CCR5 antibody MC-1, described, inter alia, in Mack (1998) J. Exp. Med. 187:1215-1224, Brühl et al., 2001, J Immunol 166(4):2420-26 and co-pending U.S. patent application Ser. No. 09/948,004, filed Sep. 5, 2001, owned by the same assignee as the present application. Other α-CCR5 antibodies, like MC-5 (as characterized in the appended examples and disclosed in Segerer (1999), loc. cit.) also may be employed in the context of this invention. The antibody specific for the T cell surface polypeptide, such as a CD3 antigen, may be selected from the group consisting of antibodies recognizing the gamma, delta, epsilon, zeta chains, such as the CD3 zeta chain (Jakobs (1997) Cancer Immunol Immunother. 44, 257-264; Mezzanzanica (1991) Cancer Res 51, 5716-5721). Examples of anti-epsilon chain antibodies are OKT3 (WO 91/09968, Kung et al., Science 206, 347-349 (1979); Van Wauwe, J. Immunol. 124, 2708-2713 (1980); Transy, Eur. J. Immunol. 19, 947-950 (1989); Woodle, J. Immunol. 148, 2756-2763 (1992); Ada, Human. Antibod. Hybridomas, 41-47 (1994)) and TR66 (Traunecker (1991) EMBO J. 10, 3655-3659). Examples of monoclonal antibodies against the CD3 zeta chain are H2D9, TIA2 (both Becton Dickinson), G3 (Serotec Ltd.).

In another embodiment of the invention, the antibody construct is a bispecific antibody which binds to a chemokine receptor as a first antigen and a toxin as a second antigen. The antibody may be covalently bound to the toxin, and the antibody-toxin construct may be constructed by chemical coupling, producing a fusion protein or a mosaic protein from the antibody and from a modified or unmodified prokaryotic or eukaryotic toxin. Furthermore, the antibody may be joined to a toxin via multimerization domains.

In a further embodiment of the present invention, the antibody construct can, via a multimerization domain, be bound in vitro and/or in vivo to a second antibody construct which binds to a CD3 antigen and/or a toxin. The multimerization may, inter alia, be obtained via hetero(di)merization. For example, the hetero(di)merization region of constant immunoglobulin domains may be employed. Other multi- and/or heterodimerization domains are known in the art and are based on leucine zippers, α- and β-chains of T-cell receptors or MHC-class II molecules. Furthermore, jun- and fos-based domains may be employed (de Kuif (1996) J. Biol. Chem. 271:7630-7634; Kostelny (1992), J. Immunol. 148, 1547-1553). Additional examples of multimerization domains are p53- and MNT-domains as described, e.g., in Sakamoto (1994) Proc. Natl. Acad. Sci. USA 91, 8974-8978; Lee (1994) Nat. Struct. Biol. 1, 877-890; Jeffrey (1995) Science 267, 1498-5102 or Nooren (1999) Nat. Struct. Biol. 6, 755-759.

In another embodiment of the invention, the chimeric polypeptide of the methods described herein is a chemokine construct, e.g., a fusion construct of a modified or an unmodified chemokine with a modified or an unmodified toxin. The construct may be bound in vitro and/or in vivo, e.g., by a multimerization domain, to an antibody construct which binds to a T cell surface polypeptide, e.g., a CD3 antigen, a dendritic cell surface polypeptide, and/or to a toxin. Suitable multimerization domains have been described in the art and are described herein. The chemokine-toxin constructs may, inter alia, result from chemical coupling, may be recombinantly produced, or may be produced as a fusion protein from a chemokine and a modified or unmodified prokaryotic or eukaryotic toxin. In one aspect, the moiety that specifically binds to a chemokine receptor, e.g., a chemokine or fragment thereof, binds to a human chemokine receptor, e.g., CCR5 or CCR4, and comprises, inter alia, RANTES, MIP-1α, MIP-1β, MCP-2, MCP-3 or (a) fragment(s) thereof which are capable of binding to the desired chemokine receptor.

The chimeric polypeptides used in the methods of the of the invention can comprise any cytotoxic agent. For example, in one aspect, the toxin may be a polypeptide toxin, e.g., a Pseudomonas exotoxin, like PE38, PE40 or PE37, or a truncated version thereof, or a ribosome inactivating protein gelonin (e.g., Boyle (1996) J. of Immunol. 18:221-230), and the like. The compositions of the invention can be conjugated to any cytotoxic pharmaceuticals, e.g., radiolabeled with a cytotoxic agents, such as, e.g., ¹³¹I (e.g., Shen (1997) Cancer 80(12 Suppl):2553-2557), copper-67 (e.g., Deshpande (1988) J. Nucl. Med. 29:217-225).

Furthermore, the chemokine construct may comprise the chemokine covalently bound to an antibody construct which binds to an antibody construct capable of binding to a T cell surface polypeptide, e.g., a CD3 antigen, and/or which is a covalently bound to a toxin.

In one embodiment, the chimeric polypeptide is an antibody and/or chemokine construct is a heterominibody construct comprising at least an antibody and/or a chemokine which binds to a chemokine receptor, such as the CCR5, CCR4 or CCR3 receptor, e.g., a human CCR5, CCR4 or CCR3 receptor. The heterominibody construct may comprise at least one toxin; in one aspect the heterominibody construct binds to the chemokine receptor as defined hereinabove and/or to a T cell surface polypeptide, e.g., a CD3 antigen, of an effector cell. Exemplary chemokines are mentioned hereinabove; exemplary toxins are described hereinabove, which may be modified or unmodified. Chemokines are well known in the art and described, inter alia, in Murphy (1999), loc. cit. Therefore, the chemokine can be selected from the group consisting of RANTES, MIP-1β, MIP-1α, MCP-2, and MCP-3 or a functional fragment thereof. In one aspect, the chemokine is RANTES. Functional fragments of chemokines are fragments which are capable of binding to or interacting with the chemokine receptor, e.g., a human CCR5. Heterominibodies are known in the art and their production is described, inter alia, in WO 00/06605. The heterominibody may be a multifunctional compound comprising at least one antibody and/or chemokine binding to or interacting with a chemokine receptor, such as human CCR5 or CCR3, may (additionally) comprise a toxin as defined herein and/or a binding site for a T cell surface polypeptide, e.g., the CD3 antigen.

In one embodiment, the antibody- or chemokine construct is a fusion (poly)peptide or a mosaic (poly)peptide. The fusion (poly)peptide may comprise merely the domains of the constructs as described herein, as well as (a) functional fragment(s) thereof. However, it is also envisaged that said fusion (poly)peptide comprises further domains and/or functional stretches. Therefore, said fusion (poly)peptide can comprise at least one further domain, this domain being linked by covalent or non-covalent bonds. The linkage as well as the construction of such constructs, can be based on genetic fusion according to the methods known in the art (e.g., Sambrook et al., loc. cit., Ausubel, “Current Protocols in Molecular Biology”, Green Publishing Associates and Wiley Interscience, N.Y. (1989)) or can be performed by, e.g., chemical cross-linking as described in, e.g., WO 94/04686. The additional domain present in the construct may be linked by a flexible linker, such as a (poly)peptide linker, wherein the (poly)peptide linker can comprises plural, hydrophilic, peptide-bonded amino acids of a length sufficient to span the distance between the C-terminal end of said further domain and the N-terminal end of the peptide, (poly)peptide or antibody or vice versa. The linker may, inter alia, be a Glycine, a Serine and/or a Glycine/Serine linker. Additional linkers comprise oligomerization domains. Oligomerization domains can facilitate the combination of two or several autoantigens or fragments thereof in one functional molecule. Non-limiting examples of oligomerization domains comprise leucine zippers (like jun-fos, GCN4, E/EBP; Kostelny, J. Immunol. 148 (1992), 1547-1553; Zeng, Proc. Natl. Acad. Sci. USA 94 (1997), 3673-3678, Williams, Genes Dev. 5 (1991), 1553-1563; Suter, “Phage Display of Peptides and Proteins”, Chapter 11, (1996), Academic Press), antibody-derived oligomerization domains, like constant domains CH1 and CL (Mueller, FEBS Letters 422 (1998), 259-264) and/or tetramerization domains like GCN4-LI (Zerangue, Proc. Natl. Acad. Sci. USA 97 (2000), 3591-3595).

Furthermore, the antibody- or chemokine construct to be used in the present methods, as described herein, may comprise at least one further domain, inter alia, domains which provide for purification means, like, e.g. histidine stretches. The further domain(s) may be linked by covalent or non-covalent bonds.

The linkage can be based on genetic fusion according to the methods known in the art and described herein or can be performed by, e.g., chemical cross-linking as described in, e.g., WO 94/04686. The additional domain present in the construct as described and disclosed in the invention may be linked by a flexible linker, such as a polypeptide linker to one of the binding site domains; the polypeptide linker can comprise plural, hydrophilic or peptide-bonded amino acids of a length sufficient to span the distance between the C-terminal end of one of said domains and the N-terminal end of the other of said domains when said polypeptide assumes a conformation suitable for binding when disposed in aqueous solution. The polypeptide linker can be a polypeptide linker as described. The polypeptide of the invention may further comprise a cleavable linker or cleavage site for proteinases, such as enterokinase

It is also envisaged that the constructs disclosed for use in the methods of the present invention can comprise further domain(s) which may function as immunomodulators. The immunomodulators comprise, but are not limited to cytokines, lymphokines, T cell co-stimulatory ligands, etc.

Adequate activation resulting in priming of naive T-cells is critical to primary immunoresponses and depends on two signals derived from professional APCs (antigen-presenting cells) like dendritic cells. The first signal is antigen-specific and normally mediated by stimulation of the clonotypic T-cell antigen receptor (TCR) that is induced by processed antigen presented in the context of MHC class-I or MHC class-II molecules. However, this primary stimulus is insufficient to induce priming responses of naive T-cells, and the second signal is required which is provided by an interaction of specific T-cell surface molecules binding to co-stimulatory ligand molecules on antigen presenting cells (APCs), further supporting the proliferation of primed T-cells. The term “T-cell co-stimulatory ligand” therefore denotes in the light of the present invention molecules, which are able to support priming of naive T-cells in combination with the primary stimulus and include, but are not limited to, members of the B7 family of proteins, including B7-1 (CD80) and 137-2 (CD86).

The chimeric polypeptides described herein may comprise further receptor or ligand function(s), and may comprise immuno-modulating effector molecule or a fragment thereof. An immuno-modulating effector molecule positively and/or negatively influences the humoral and/or cellular immune system, particularly its cellular and/or non-cellular components, its functions, and/or its interactions with other physiological systems. The immuno-modulating effector molecule may be selected from the group consisting of cytokines, chemokines, macrophage migration inhibitory factor (MIF; as described, inter alia, in Bernhagen (1998), Mol Med 76(3-4); 151-61 or Metz (1997), Adv Immunol 66, 197-223), T-cell receptors and soluble MHC molecules. Such immuno-modulating effector molecules are well known in the art and are described, inter alia, in Paul, “Fundamental immunology”, Raven Press, New York (1989). In particular, known cytokines and chemokines are described in Meager, “The Molecular Biology of Cytokines” (1998), John Wiley & Sons, Ltd., Chichester, West Sussex, England; (Bacon (1998). Cytokine Growth Factor Rev 9(2):167-73; Oppenheim (1997). Clin Cancer Res 12, 2682-6; Taub, (1994) Ther. Immunol. 1(4), 229-46 or Michiel, (1992). Semin Cancer Biol 3(1), 3-15).

The constructs described herein may comprise domains originating from one species, e.g., from mammals, such as human. However, chimeric and/or humanized constructs are also envisaged and within the scope of the present invention.

In one embodiment, the construct of the invention comprises a cross-linked (poly)peptide construct. As described herein, the cross-linking may be based on methods known in the art, which comprise recombinant as well as biochemical methods.

In embodiment of the present invention, the chimeric polypeptide, e.g., an antibody construct or a chemokine construct, comprises at least one toxin. The toxin may be Pseudomonas exotoxin A, diphtheria toxin and similar toxins. It is envisaged that truncated toxins are employed, like the PE38 or the PE40 of Pseudomonas toxin described in the appended examples. The toxin may be bound to said antibody or chemokine by means as described herein. It is also envisaged that said toxin is bound to the chimeric polypeptide (e.g., antibody/chemokines) by means of a short peptide linker. The linker can comprise a flexible and hydrophilic amino acid sequence, e.g., of glycines and serines. The linker can has a length of 1 to about 20 amino acids, or more.

Several fusion proteins with a truncated version of Pseudomonas exotoxin A have been designed. Most of them have been used to target and destroy malignant cells. This toxin becomes activated upon proteolytic cleavage. A truncated version of the toxin (PE38) may be employed for the constructs of the present invention, as the full-length protein binds with its fist domain to the ubiquitous α2-macroglobulin receptor and is therefore toxic to most eukaryotic cells. Yet, this problem may be overcome by replacing the first domain of Pseudomonas exotoxin A by a specific sequence in order to alter the binding specificity of the toxin.

Exemplary chimeric polypeptides useful in the present methods, therefore, include (a) antibody constructs comprising a binding site for CCR5 and a binding site for CD3, and (b) chemokine constructs comprising RANTES and a toxin, e.g., the truncated Pseudomonas exotoxin A (PE38).

Construction and purification of the chimeric polypeptides described herein as useful in the present methods is within the skill of an ordinary artisan. More detailed guidance is provided in co-pending U.S. patent application Ser. No. 09/948,004, filed Sep. 5, 2001, owned by the same assignee as the present application.

Pharmaceutical Compositions and Administration

The methods described herein include administering at least one chimeric polypeptide polynucleotide, vector, host cell, (e.g., antibody construct and/or chemokine construct and corresponding vectors and host cells) described herein. The composition, optionally, further comprises one or more other molecules, either alone or in combination, that are useful to manage or treat asthma, e.g., anti-inflammatory agents, corticosteroids, bronchodilators, anti-leukotrienes, and anti-asthma therapeutic antibodies. The composition may be in solid, liquid or gaseous form and may be, inter alia, in a form of a powder(s), a tablet(s), a solution(s) or an aerosol(s). In alternative embodiments, the composition comprises at least two, at least three, at least four, or more than four, chimeric polypeptides described herein. The composition can be a pharmaceutical composition further comprising, optionally, a pharmaceutically acceptable carrier, diluent and/or excipient. A preferred carrier, diluent and/or excipient is one formulated for intratracheal delivery.

Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well known conventional methods. These pharmaceutical compositions can be administered to the subject at a suitable dose. Administration of the suitable compositions may be effected by different ways, preferably by intranasal, intratracheal or intrabronchial administration, e.g., by inhalation or aerosol administration, e.g., with the use of an inhaler (e.g., a metered dose inhaler), a spacer, or a nebulizer. Administration can also be effected by oral intravenous, intraperitoneal, subcutaneous, intramuscular, topical, or intradermal administration.

The dosage regiment can be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Generally, the regimen as a regular administration of the pharmaceutical composition should be in the range of about 1 ng to 500 μg, preferably about 10 ng to 250 μg, of the chimeric polypeptide per kg of body weight, depending on the mode of administration. For administration by inhalation or intranasally, the dosage will generally be lower than that used with subcutaneous or intravenous administration. For example, for intravenous or subcutaneous administration, the unit dose can be, e.g., between 1 μg-150 μg per kg of body weight, preferably between 1 μg-70 μg per kg of body weight, more preferably between about 1 μg-25 μg per kg of body weight. For intranasal or intrabronchial administration the unit dose can be between 10 ng-1.5 μg per kg of body weight, preferably between 10 ng-700 ng per kg of body weight, more preferably between 10 ng-500 ng per kg of body weight, most preferably between 10 ng-250 ng per kg of body weight, or between 10 ng-100 ng per kg of body weight. In some circumstances, less than 10 ng of the chimeric polypeptide per kg will be an appropriate dose. In some embodiments, a preferred dose for intranasal or intrabronchial administration is less than 1 μg/kg.

In some embodiments, the chimeric polypeptide is administered in an amount effective to provide a plasma level of the polypeptide in a subject of between 1 and 100 μg/ml, preferably between 2 and 50 μg/ml, more preferably between 5 and 15 μg/ml. Unit dosages for children can be correspondingly lower.

If the regimen is a continuous infusion, it can also be in the range of about 1 ng to 10 μg units per kilogram of body weight per minute. An alternative dosage for continuous infusion may be in the range of about 0.01 μg to 10 μg per kilogram of body weight per hour. Other exemplary dosages are recited herein below. For example, a dosage for intravenous or intrabronchial administration of DNA can be from approximately 10⁶ to 10¹² copies of the DNA molecule.

A unit dose can be administered more than once. For example, a unit dose of a chimeric polypeptide can be administered two, three, four, five, six, ten, times or more, e.g., after exposure or suspected exposure or impending exposure of the subject (e.g., human subject) to an allergen to which the subject is hypersensitive. The multiple administrations can take place, e.g., no more than once every 6 weeks, once every 4 weeks, once every 2 weeks, once every 10 days, once every 7 days, or even daily. For example, a unit dose can be administered once a day for a specified period, e.g., once a day for a period of at least one week, 2 weeks, one month, 2 months, 3 months, 4 months, 6 months, 9 months, a year, two years or more.

Progress can be monitored by periodic assessment. For example, IgE levels, airway hyperresponsiveness, coughing, wheezing, chest tightness, dyspnea, airway smooth muscle contraction, bronchial mucus secretion, inflammation, vasodilation, recruitment of inflammatory cells (e.g., neutrophils, monocytes, macrophages, lymphocytes, eosinophils) to inflammation sites, goblet cell hyperplasia, release of inflammatory mediators by mast cells or migrating inflammatory cells, or other criteria known to an ordinary practitioner, can be assessed.

The chimeric polypeptide may be administered locally or systematically. Administration can be parenterally, e.g., intravenously; and, by external administration, e.g., by inhalation. DNA may also be administered directed to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, anti-microbials, antioxidants, chelating agents, and inert gases and the like. In addition, the pharmaceutical composition of the present invention may comprise proteinaceous carriers, like, e.g., serum albumin or immunoglobulin, including those of human origin.

Furthermore, it is envisaged that the pharmaceutical composition of the invention may comprise further biologically active agents, e.g., one or more corticosteroid, bronchodilator, anti-inflammatory agent, therapeutic antibody, leukotriene antagonist or other anti-asthma therapeutic, e.g., an anti-asthma therapeutic described herein.

Cell and Gene Therapy

In one aspect, polynucleotides and vectors encoding the chimeric polypeptides described herein are administered either alone or in any combination using standard vectors and/or gene delivery systems, and, optionally, together with a pharmaceutically acceptable carrier or excipient. Subsequent to administration, the polynucleotides or vectors may be stably integrated into the genome of the subject, such as a human.

Alternatively, pharmaceutical compositions, including, e.g., vectors, such as viral vectors, of the invention are designed to be specific for (e.g., “target to”) certain cells or tissues; they can also be designed to persist in cells. Suitable pharmaceutical carriers and excipients are well known in the art.

Furthermore, it is possible to use a pharmaceutical composition described herein comprising a polynucleotide or vector encoding a chimeric polypeptide in gene therapy. Suitable gene delivery systems may include liposomes, receptor-mediated delivery systems, naked DNA, and viral vectors such as herpes viruses, retroviruses, adenoviruses, and adeno-associated viruses, among others. Delivery of nucleic acids to a specific site in the body for gene therapy may also be accomplished using a biolistic delivery system, such as that described by Williams (Proc. Natl. Acad. Sci. USA 88 (1991), 2726-2729). Further methods for the delivery of nucleic acids comprise particle-mediated gene transfer as, e.g., described in Verma, Gene Ther. 15 (1998), 692-699. Gene therapy approaches for targeting cells of the airway are described, e.g., in Johnson, Ann N Y Acad Sci 2001 Dec;953:43-52 and Davies et al., Curr Opin Pharmacol 2001 Jun;1(3):272-7.

It is to be understood that the introduced polynucleotides and vectors express a gene product after introduction into the cell; they can remain in this status during the lifetime of the cell. For example, cell lines that stably express the polynucleotide under the control of appropriate regulatory sequences may be engineered according to methods well known to those skilled in the art. Rather than using expression vectors that contain viral origins of replication, host cells can be transformed with the polynucleotide of the invention and a selectable marker, either on the same or separate plasmids. Following the introduction of foreign DNA, engineered cells may be allowed to grow for about 1 to 2 days in an enriched media, and then are switched to a selective media. The selectable marker in the recombinant plasmid confers resistance to the selection. This allows for the selection of cells having stably integrated the plasmid into their chromosomes; the selected cells grow to form foci, which, in turn, can be cloned and expanded into cell lines.

A number of selection systems may be used, including but not limited to, the herpes simplex virus thymidine kinase (Wigler, Cell 11 (1977), 223), hypoxanthine-guanine phosphoribosyltransferase (Szybalska, Proc. Natl. Acad. Sci. USA 48 (1962), 2026), and adenine phosphoribosyltransferase (Lowy, Cell 22 (1980), 817) in tk-, hgprt- or aprt- cells, respectively. Also, antimetabolite resistance can be used as the basis of selection for dhfr, which confers resistance to methotrexate (Wigler, Proc. Natl. Acad. Sci. USA 77 (1980), 3567; O'Hare, Proc. Natl. Acad. Sci. USA 78 (1981), 1527), gpt, which confers resistance to mycophenolic acid (Mulligan, Proc. Natl. Acad. Sci. USA 78 (1981), 2072); neo, which confers resistance to the aminoglycoside G-418 (Colberre-Garapin, J. Mol. Biol. 150 (1981), 1); hygro, which confers resistance to hygromycin (Santerre (1984) Gene 30:147); or puromycin (pat, puromycin N-acetyl transferase). Additional selectable genes have been described, e.g., trpB, which allows cells to utilize indole in place of tryptophan, hisD, which allows cells to utilize histinol in place of histidine (Hartman, Proc. Natl. Acad. Sci. USA 85 (1988), 8047); and ODC (omithine decarboxylase) which confers resistance to the omithine decarboxylase inhibitor, 2-(difluoromethyl)-DL-ornithine, DFMO (McCologue, 1987, In: Current Communications in Molecular Biology, Cold Spring Harbor Laboratory ed.).

In a further embodiment, the invention relates to a medicament, such as a pharmaceutical composition comprising a chimeric polypeptide described herein, for the treatment or prevention of allergic airway disease, e.g., allergic asthma, e.g., chronic allergic asthma.

The chimeric polypeptides described herein (e.g., bispecifc antibodies) are useful in specifically destroying chemokine receptor-positive cells. For example, a bispecific antibody, binding simultaneously to CCR5 on target cells and to CD3 on T-cells, redirects cytotoxic T-cells to the CCR5 positive target cells.

Other chimeric polypeptides, such as the chemokine constructs described herein (for example, a fusion protein of the chemokine RANTES and a truncated version of the Pseudomonas exotoxin A (PE38)), are able to bind to CCR5 or CCR4 and to downmodulate the receptor from the cell surface.

The present invention also provides for a kit comprising a polynucleotide, a vector, a host cell, a chimeric polypeptide (e.g., an antibody construct and/or a chemokine construct) as described herein. The kit of the invention may further comprise a storage solution(s) and/or other reagents or materials required for the conduct of scientific or therapeutic methods. The kit may, inter alia, comprise drugs and/or medicaments employed in the treatment of allergic asthma. Furthermore, parts of the kit of the invention can be packaged individually in vials or bottles or in combination in containers or multicontainer units. The kits comprise instructions for using the chimeric polypeptides described herein in the treatment or prevention of allergic airway disease, e.g., allergic asthma, e.g., chronic allergic asthma.

### EXAMPLES

### Example 1

Temporal Changes in Whole Lung Levels of RANTES/CCL5 Protein Levels Following Conidia Challenge in A. fumigatus-Sensitized Mice

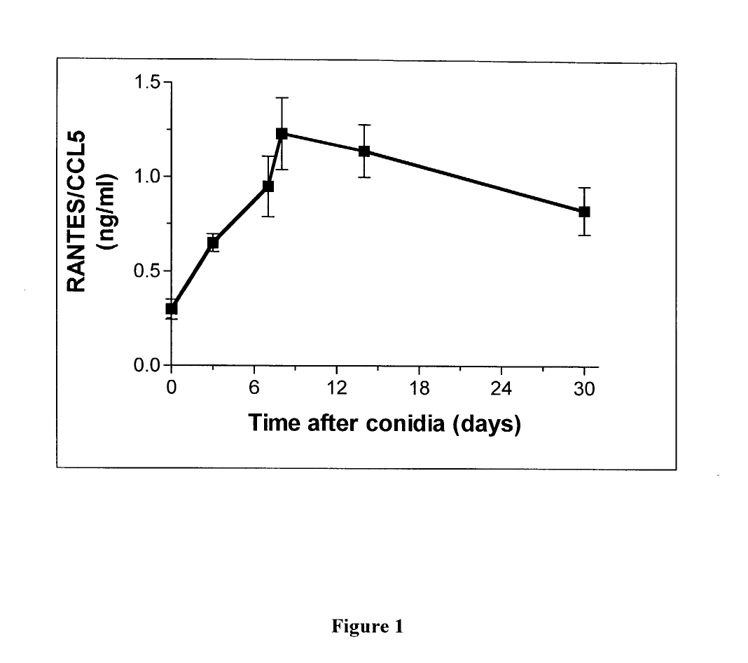

In this example, it was determined whether RANTES/CCL5 levels in bronchoalveolar lavage (BAL) and whole lung samples were altered during the course of chronic allergic airway disease. Several time points were examined in order to develop a detailed picture of the temporal changes in this chemokine over the 30 days following conidia introduction into A. fumigatus-sensitized mice. ELISA analysis of RANTES/CCL5 in whole lung homogenates from A. fumigatus-sensitized mice challenged with A. fumigatus conidia are shown in FIG. 1. At all times examined after the conidia challenge, whole lung levels of RANTES/CCL5 were significantly (P≦0.05) increased above levels measured immediately prior to the conidia challenge (i.e. baseline levels; FIG. 1). The highest levels of RANTES/CCL5 were detected at day 8 after the conidia challenge. RANTES/CCL5 was not detected in the BAL prior to the conidia challenge (i.e. day 0) and BAL levels of this CC chemokine were typically below the limits of detection at various times after the conidia challenge (not shown). Thus, these data show that whole lung levels of RANTES/CCL5 were increased in A. fumigatus-sensitized mice after an intratracheal challenge with A. fumigatus conidia.

### Example 2

Temporal Changes in BAL and Whole Lung Levels of RANTES/CCL5 Protein Levels Following Ad RANTES and Conidia Challenge in A. fumigatus-Sensitized Mice

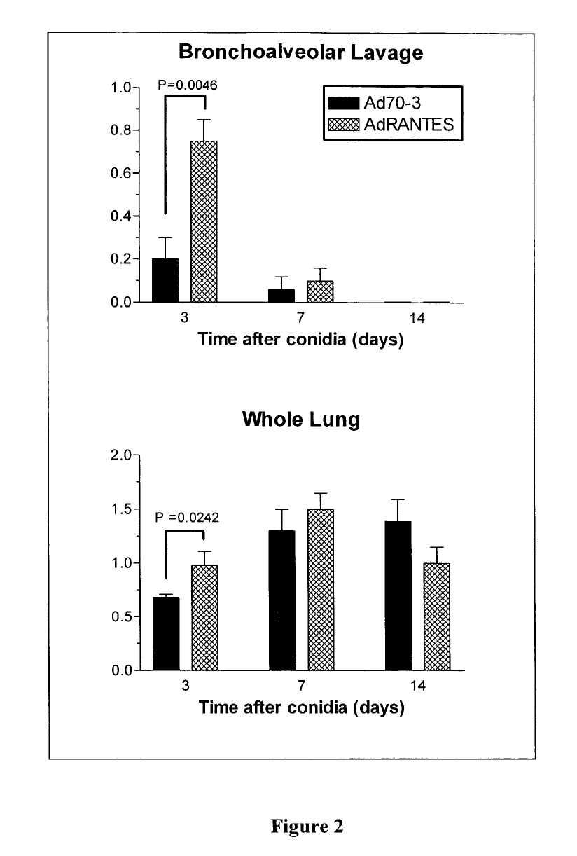

In this example it was determined whether adenovirus-mediated overexpression of murine RANTES/CCL5 (AdRANTES) altered any of the features of this allergic airway disease model. Previous studies showed that the intratracheal administration of AdRANTES induced the expression of RANTES/CCL5 in the bronchial epithelium and significantly increased RANTES/CCL5 levels in the lungs of naive rats for up to 7 days after adenovirus administration (32). As shown in FIG. 2, the presence of AdRANTES significantly increased the immunoreactive levels of RANTES/CCL5 in BAL samples (top panel, FIG. 2) and whole lungs (bottom panel, FIG. 2) compared with levels of this chemokine measured in BAL and lung samples from mice exposed to Ad70-3 at day 3 after the conidia challenge. However at days 7 and 14 after conidia, no differences in RANTES/CCL5 levels in either lung compartment were observed between the two-adenovirus groups. Thus, the presence of AdRANTES significantly, but transiently, increased RANTES/CCL5 levels in the BAL and whole lung during the conidia challenge period in A. fumigatus-sensitized mice.

### Example 3

Adenoviral-Mediated Over Expression of RANTES/CCL5 Markedly Augmented the Airway Hyperresponsiveness Associated with Chronic Allergic Airway Disease

This example examines the effect of RANTES/CCL5 overexpression in chronic allergic airway disease.

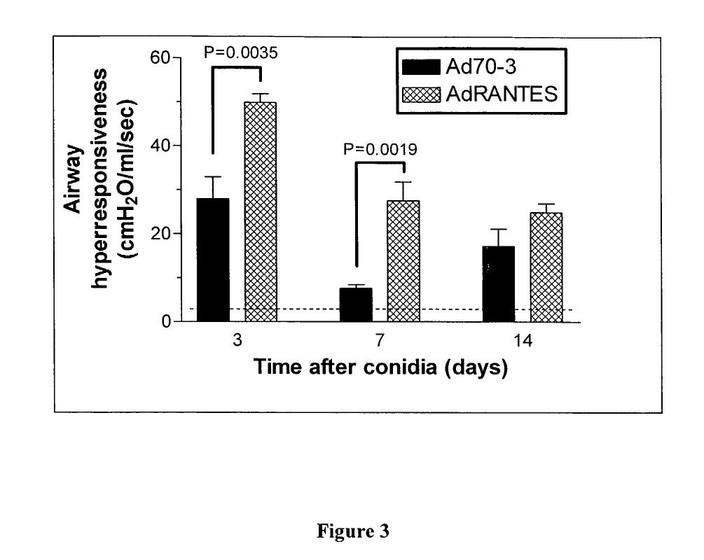

Although several studies have documented that RANTES/CCL5 levels are significantly increased during clinical asthma, the relative contribution of this chemokine to changes in airway responsiveness is still undefined. Experimental studies to date have not completely resolved the role of RANTES/CCL5 in this response either, as RANTES/CCL5 contributes to the airway hyperreactivity associated with A. fumigatus (29) and OVA (28) but not that associated with S. mansoni egg antigen (27). A. fumigatus-sensitized mice that received the AdRANTES vector at the time of the bolus intratracheal conidia challenge exhibited significantly increased methacholine-induced airway hyperresponsiveness at days 3 and 7 of this model compared with similarly sensitized and challenged mice that received Ad70-3 and conidia (FIG. 3). At day 14 after conidia and adenovirus challenge, airway hyperreactivity following methacholine provocation was similar in both groups. These data showed that AdRANTES-induced expression of RANTES/CCL5 transiently but significantly enhanced airway hyperresponsiveness associated with Aspergillus-induced allergic airway disease.

Previous studies on the impact of AdRANTES instillation into non-sensitized, non-allergic rats showed that the intratracheal administration of this viral vector targeted the transient expression of RANTES/CCL5 to the bronchial epithelium of the lung (32). In this previous study, AdRANTES treatment in rats increased the number of macrophages, monocytes, and neutrophils in BAL samples, and the greatest effect appeared to be on monocyte numbers. However, the increased recruitment of monocytes appeared to be transitory since by day 7 after the AdRANTES challenge, numbers of these cells were decreased while cosinophil and lymphocyte numbers remained elevated at this time (32). In the data described herein, it was observed that immunoreactive levels of RANTES/CCL5 were significantly increased at day 3, but not at other times, after conidia and AdRANTES challenge in A. fumigatus-sensitized mice compared with similarly sensitized mice that received conidia and the control adenovirus vector Ad70-3. In agreement with the findings of Braciak et al (32), a similar pattern in leukocyte recruitment into the BAL of AdRANTES- and conidia-challenged mice previously sensitized to A. fumigatus was observed. Specifically, mononuclear cells were significantly increased in the BAL at day 3 after AdRANTES and conidia challenge, whereas eosinophil and lymphocyte numbers in the BAL were significantly increased at day 7 after AdRANTES and conidia challenge.

### Example 4

Adenoviral-Mediated Overexpression of RANTES/CCL5 in the Lung Markedly Augmented the Airway Accumulation of Eosinophils and Lymphocytes After Conidia Challenge in A. fumigatus-Sensitized Mice

This example examined whether the enhanced airway hyperresponsiveness observed at days 3 and 7 in AdRANTES-treated mice was due, in part, to the chemotactic effect of RANTES/CCL5 on eosinophil and lymphocyte recruitment to the airways (28).

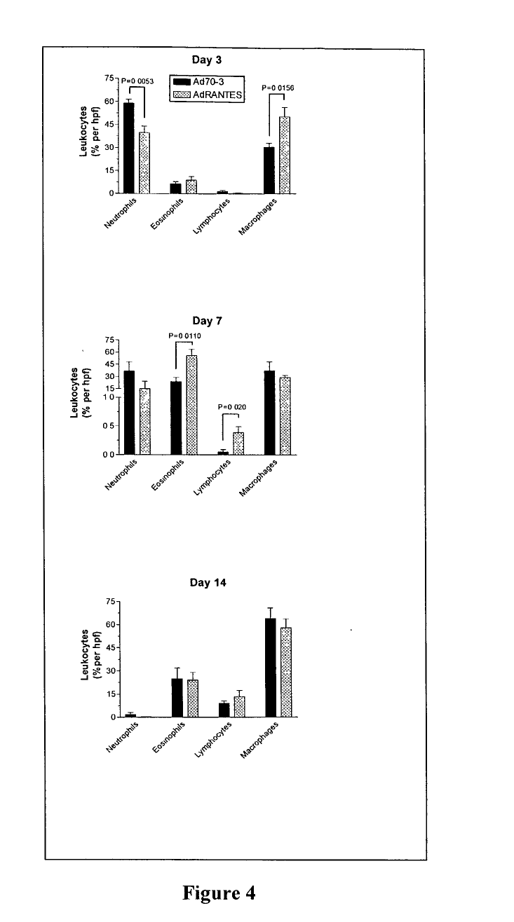

As shown in FIG. 4, significant differences in neutrophil and macrophage counts in the BAL were observed between the two adenovirus-treated groups at day 3 after conidia. Specifically, the numbers of neutrophils were significantly decreased whereas macrophage numbers were significantly increased in the AdRANTES group compared with the Ad70-3 group. At day 7 after adenovirus and conidia challenge, significant increases in cosinophil and lymphocyte numbers in the BAL of AdRANTES-treated mice were observed. All leukocyte population counts in the BAL were similar in both groups of mice at day 14 (FIG. 4).

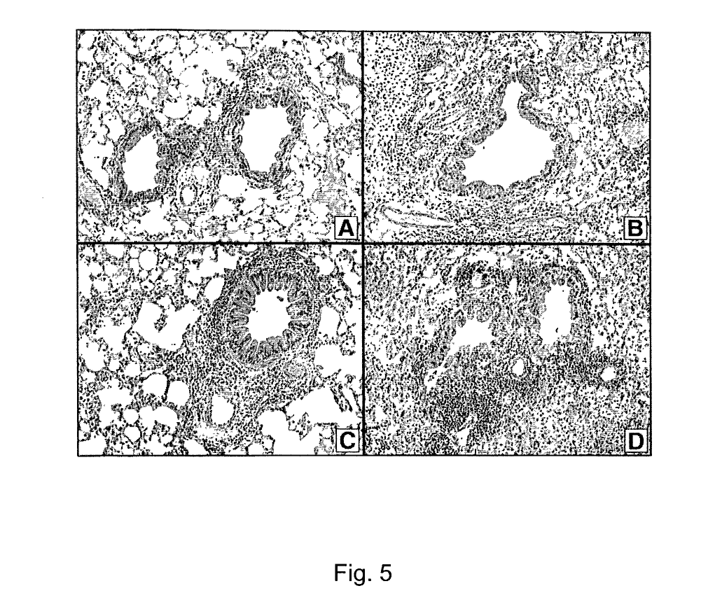

The significance of changes in numbers of leukocytes in the BAL has been called into question by various investigators who have shown no correlation between leukocytes in the BAL and changes in airway hyperresponsiveness (28, 37). We have also previously observed a similar discordance in the chronic allergic airway disease model (38). In contrast, changes in the numbers of inflammatory leukocytes around the airways appear to closely correlate with the enhancement or inhibition of airway hyperreactivity (28). Representative histological assessment from both groups of mice at various times after conidia is summarized in FIG. 5. At days 3 (Panels A & B) and 7 (Panels C & D) after conidia, markedly greater numbers of eosinophils and lymphocytes were detected in the lungs and around the airways of AdRANTES-treated mice compared with the Ad70-treated groups. The transgene-induced expression of RANTES/CCL5 enhanced the peribronchial accumulation of eosinophils and lymphocytes around the airways of A. fumigatus mice challenged with A. fumigatus conidia.

### Example 5

Adenoviral-Mediated Overexpression of RANTES/CCL5 Significantly Augmented Whole Lung Levels of MDC/CCL22.

This example examined whether AdRANTES altered the levels of this chemokine during chronic allergic airway disease due to Aspergillus. Gonzalo et al. (28) demonstrated that RANTES/CCL5 regulated eotaxin/CCL11 production in the context of OVA-induced allergic airway disease.

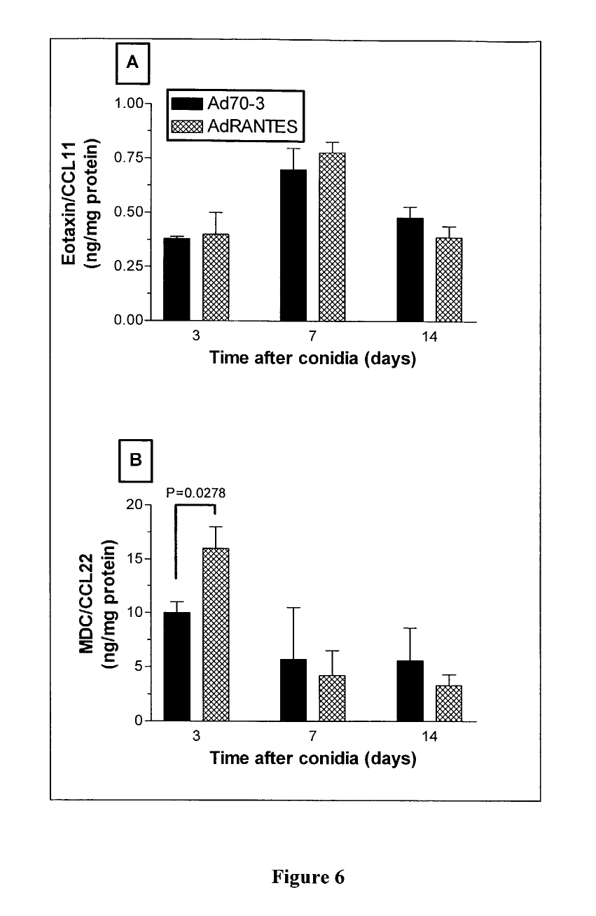

At all times examined after adenovirus and conidia challenge in A. fumigatus-sensitized mice whole lung levels of eotaxin/CCL11 did not differ between the two groups (FIG. 6A). However, ELISA analysis of MDC/CCL22 showed that the AdRANTES treatment significantly increased the immunoreactive levels of this chemokine in whole lung samples (FIG. 6B). Interestingly, MDC/CCL22 was recently shown to promote the peribronchial retention of eosinophils and contribute to airway hyperreactivity during experimental OVA-induced allergic airway disease (37). Thus, these data demonstrated that the increased expression of RANTES/CCL5 in A. fumigatus-sensitized mice during the first week of the conidia challenge amplified the generation of another CC chemokine that has major effects on the development of allergic airway responses.

Thus, significant increases in airway hyperresponsiveness were observed in the AdRANTES-treated groups compared with the Ad70-3-treated groups at days 3 and 7 after the conidia challenge. Increased airway responses to methacholine in the AdRANTES-treated group at days 3 and 7 after conidia presumably reflect the increased numbers of eosinophils and lymphocytes that were present in and around the airways at these times. The increased accumulation of eosinophils and lymphocytes in the bronchial submucosal, but not airways (see above), directly contributes to airway hyperresponsiveness in a number of allergic airway disease models (4) (26) (56) including those induced by Aspergillus (57-59). It was surprising that the transgene-induced expression of RANTES/CCL5 significantly increased the whole lung levels of MDC/CCL22, another CC chemokine involved in airway hyperreactivity and lung inflammation (37), at day 3 after challenge. This increase induced by AdRANTES was unique to MDC/CCL22 since eotaxin/CCL11 and other CC chemokine levels were not altered by the RANTES/CCL5 transgene. Thus, the increased airway hyperresponsiveness and inflammatory leukocyte accumulation around the airways were both associated with ectopic RANTES/CCL5 expression in the lung, possibly due to its effects on the endogenous production of MDC/CCL22.

### Example 6

RANTES-PE38 Treatment Significantly Attenuated Airway Hyperresponsiveness in Mice with Aspergillus-Induced Allergic Airway Disease.

This example addressed whether targeting RANTES/CCL5-responsive cells in the lung prevented reversed the appearance of many of the features of this disease.

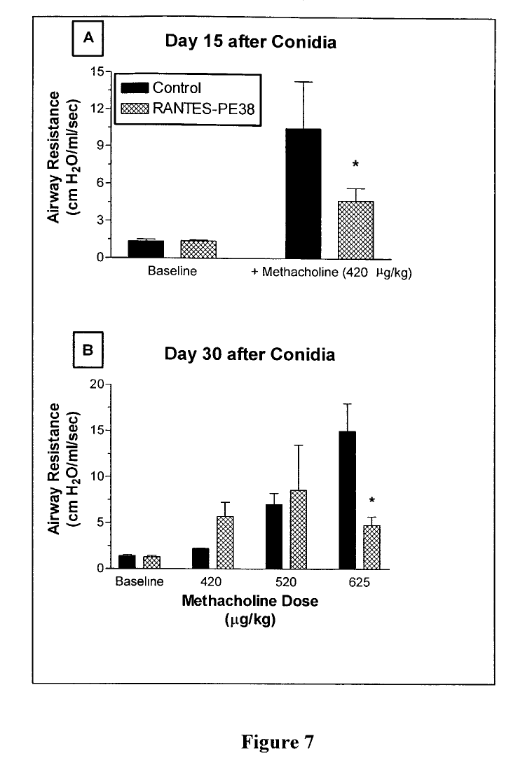

The targeting of RANTES/CCL5 in the lungs of allergic mice was facilitated by the intranasal instillation of RANTES-PE38 (30). Airway hyperreactivity in RANTES-PE38- and control-treated mice at days 15 and 30 after conidia is shown in FIG. 7. When A. fumigatus-sensitized CBA/J mice challenged with conidia received RANTES-PE38 from day 0 to day 15 after conidia, airway hyperresponsiveness to a systemic methacholine challenge of 420 μg/kg was significantly lower than that measured in similarly sensitized and challenged control mice that received the same dose of methacholine (FIG. 7A). RANTES-PE38 treatment from day 15 to 30 after conidia also significantly inhibited airway hyperresponsiveness, but this inhibitory effect was only observed in this group of mice when they received a 625 μg/kg dose of methacholine (FIG. 7B). These data suggested that the early targeting of RANTES/CCL5responsive cells in the lungs has a major effect on the development of airway hyperreactivity during chronic allergic airway disease. In addition, targeting RANTES/CCL5-responsive cells from days 15 to 30 after conidia revealed that the role of RANTES/CCL5 in airway hyperresponsiveness might be more limited at the later stages of this model.

### Example 7

RANTES-PE38 Treatment Significantly Inhibited Peribronchial Inflammation.

This example shows RANTES-PE38 effect on peribronchial inflammation.

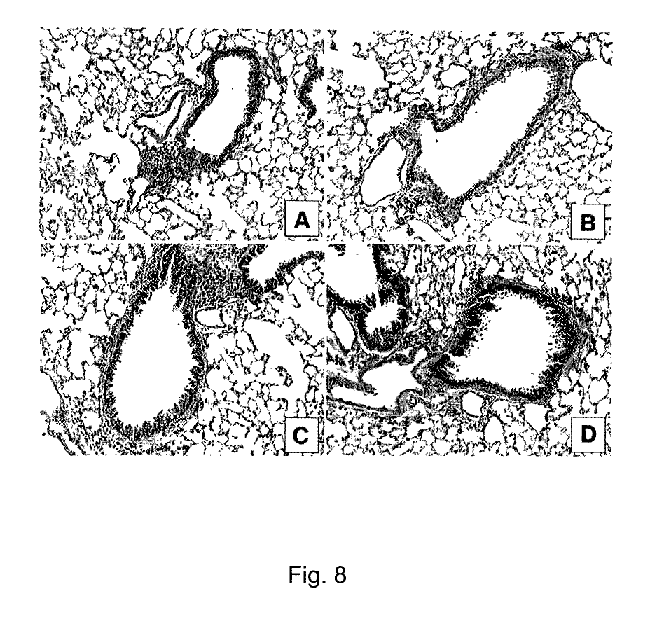

Representative histological lung sections from control and RANTES-PE38-treated mice at day 15 after conidia are shown in FIG. 8, and it was apparent that mice that received the RANTES/CCL5 chimeric protein (FIG. 8A) over the first 15 days of this model had markedly less peribronchial inflammation than control mice over the same time course (FIG. 8B). Histological analysis of whole lung sections from mice treated with saline (FIG. 8C) or RANTES-PE38 (FIG. 8D) from days 15 to 30 after conidia revealed a similar marked reduction in peribronchial inflammation at day 30. Interestingly, the diminished numbers of inflammatory leukocytes around the airways differed from what was detected in BAL samples at day 15 after conidia (Table II). Morphometric analysis of neutrophils, eosinophils, lymphocytes and macrophages revealed that significantly greater numbers of eosinophils and lymphocytes were present in BAL samples in the RANTES-PE38 group compared with the control group. BAL samples from both groups of mice at day 30 after conidia are also summarized in Table II, and fewer neutrophils and eosinophils, and significantly fewer lymphocytes were counted in samples from RANTES-PE38-treated mice compared with the control group. Similar numbers of macrophages were detected in BAL samples from both groups. These data suggested that the targeting of RANTES/CCL5-responsive cells in the lung prevented the initiation of chronic allergic airway disease, presumably because of the inability of inflammatory leukoctyes to localize around the airways of conidia-challenged mice.

### Example 8

RANTES-PE38 Treatment Inhibited Serum Levels of Total IgE.

This example shows that RANTES-PE38 treatment inhibited serum levels of total IgE.

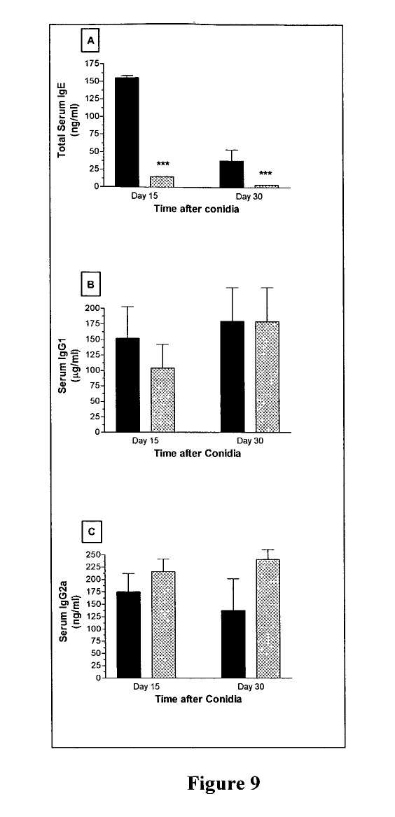

The levels of total IgE in the serum of RANTES-PE38 treated mice at days 15 and 30 after conidia were significantly lower than levels measured in the saline-treated groups at the same times after the conidia challenge (FIG. 9A). The RANTES-PE38 treatments did not affect the circulating levels of IgG1 (FIG. 9B). While it appeared that the RANTES-PE38 treatment increased IgG2A (FIG. 9C) at both times examined after the conidia challenge these differences did not reach statistical significance. Thus, RANTES-PE38 appeared to target IgE-producing B cells thereby significantly reducing circulating levels of this Ig isotype.

### Example 9

RANTES-PE38 Treatment Significantly Modulated Whole Lung Gene Expression for Chemokines and Chemokine Receptors During Aspergillus-Induced Allergic Airway Disease.

This example examined the effect of RANTES-PE38 treatment on whole lung gene expression for chemokines and chemokine receptors during Aspergillus-induced allergic airway disease.

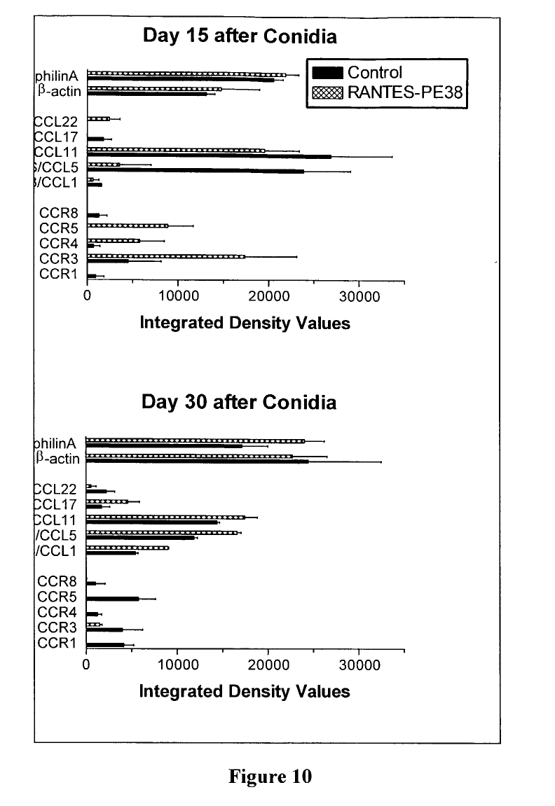

Select chemokine and chemokine receptor gene expression was evaluated using SuperArray analysis of RNA in whole lung samples at days 15 and 30 after conidia is summarized in FIG. 10. This gene analysis was included in order to examine the overall impact of RANTES-PE38 on the concomitant expression of chemokines and their receptors in the lung. In whole lung samples from the RANTES-PE38 treatment group at day 15 after conidia, gene expression for CCR3, CCR4 and CCR5 were upregulated whereas gene expression for CCR1 and CCR8 were downregulated compared with similar chemokine receptor gene expression in the control group. Further, the gene expression for RANTES/CCL5, TCA3/CCL1, and TARC/CCL17 were markedly lower in the lungs of the RANTES-PE38-treated group compared with the control group at day 15. MDC/CCL22 gene expression, while not detected in control lungs at day 15, was prominently expressed in RANTES-PE38-treated lungs at this time after conidia. At day 30 after conidia, gene expression for CCR1, CCR4, CCR5 and CCR8 were absent, and CCR3 gene levels were markedly lower, in the lungs of the RANTES-PE38-treated mice whereas gene expression for all of these CC chemokine receptors were prominently expressed in control lungs. Of the chemokine ligands shown in FIG. 10, only MDC/CCL22 gene levels were lower in the RANTES-PE38 treated group compared with the control group. Taken together, these data show that gene expression for chemokines and their corresponding chemokine receptors were markedly altered by the specific targeting of RANTES/CCL5-responsive cells in the lung during allergic airway disease.

Thus, analysis of RNA expression in whole lung samples of various CC chemokine receptors suggested that all of the major receptors that can bind RANTES/CCL5 were affected by the RANTES-PE38 treatments. Paradoxically, the levels of RNA expression for CCR3, CCR4 and CCR5 were markedly increased in whole lung samples from RANTES-PE38-treated mice at day 15 after conidia. The increase in the gene expression of these three RANTES/CCL5 receptors may reflect a compensatory response by the lung due of the specific targeting of RANTES/CCL5-responsive cells, but it is not known whether the increased gene expression for these chemokine receptors was translated into functional receptor. In contrast, gene expression of another RANTES/CCL5 receptor, CCR1, and the TCA-3/CCL1 and TARC/CCL17 receptor, CCR8, were absent in RANTES-PE38-treated mice at this time after conidia. If functional receptors for both CCR1 and CCR8 were absent or markedly reduced in RANTES-PE38-treated mice at day 15 after conidia, this would have a significant impact on many parameters of allergic airway disease including airway inflammation, hyperreactivity and remodeling (38) (64)(JMS and CMH, unpublished findings). At day 30 after conidia, CCR1, CCR3, CCR4, CCR5 and CCR8 gene expression were markedly lower or absent in RANTES-PE38-treated mice compared with the control group at this time. Once again, the absence of functional CCR1 and CCR5 could profoundly affect the persistence of allergic airway disease given that we have shown previously that A. fumigatus-sensitized CCR5−/− mice do not exhibit airway inflammation, hyperreactivity or remodeling at day 30 after conidia (29) whereas similarly sensitized and conidia challenged CCR1−/− mice have diminished airway remodeling at this time (38).

### Example 10

RANTES-PE38 Treatment Significantly Inhibits Whole Lung Levels of MDC/CCL22 and TARC/CCL17

The SuperArray analysis described above suggested that gene expression of a number of chemokines was modulated by the RANTES-PE38 treatments. To address whether these gene changes were accompanied by changes in the corresponding protein, whole lung chemokine levels were examined by specific ELISA at both time points after the conidia challenge in this example.

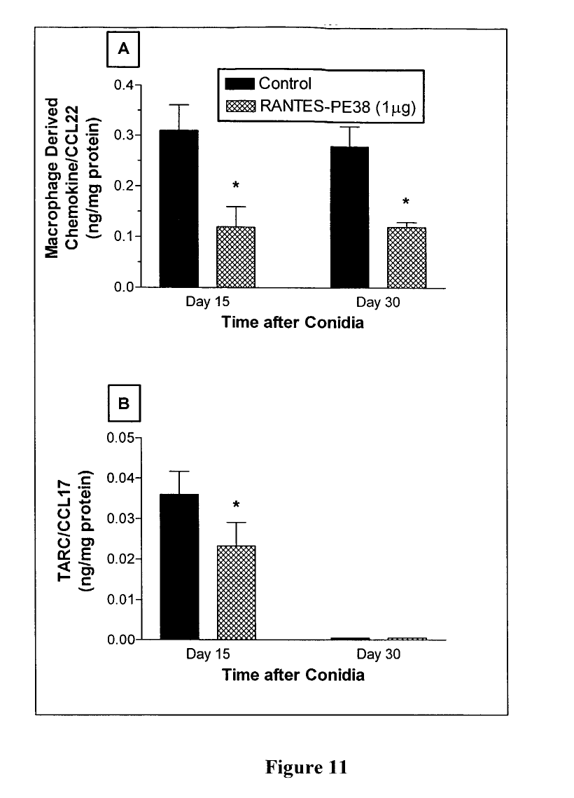

We observed that the RANTES-PE38 treatments did not affect whole lung levels of RANTES/CCL5, eotaxin/CCL11 or TCA-3/CCL1 protein at either time after conidia compared with whole lung chemokine protein levels in the appropriate control group. However, the protein levels of RANTES/CCL5 in whole lung samples from both RANTES-PE38-treated groups may have been exaggerated by the presence of RANTES-PE38, which could be detected with the ELISA system employed in this study. As shown in FIG. 11, RANTES-PE38 treatment significantly reduced whole lung levels of MDC/CCL22 (at both time points) and TARC/CCL17 (at day 15 after conidia) compared with levels of these chemokines in the appropriate control groups. These data suggested that RANTES-PE38 targeted directly or modulated the influx of cells into the lung that generated these CCR4 and CCR8 ligands.

### Example 11

Early RANTES-PE38 Treatment Attenuated Goblet Cell Hyperplasia.

This example examined goblet cell hyperplasia.

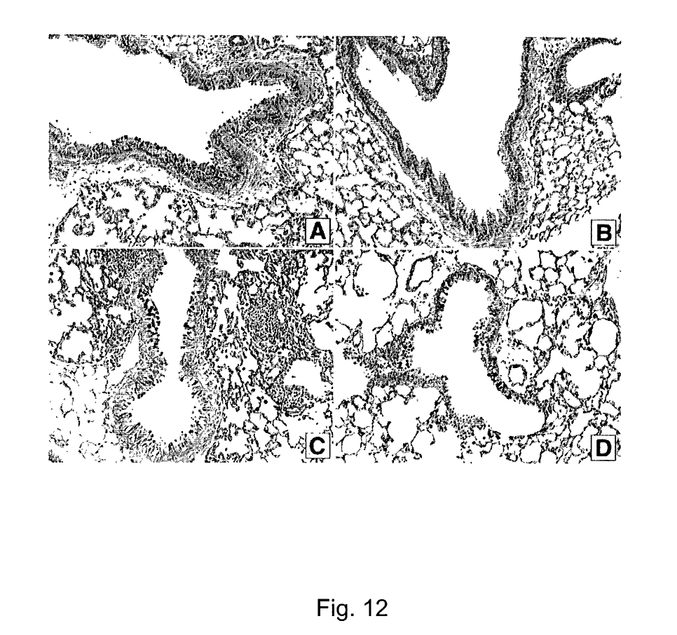

Goblet cell hyperplasia is a prominent feature of chronic allergic airway disease (31), and our previous studies have shown that the appearance of goblet cells in the airways of Aspergillus-challenged mice is CCR5- and RANTES-dependent (29). PAS staining of whole lung sections at days 15 and 30 after conidia revealed that the RANTES-PE38 treatment abolished the goblet cell hyperplasia associated with this model of chronic asthma at day 15 but not at day 30 after conidia (FIG. 12). The persistence of goblet cell hyperplasia in the latter RANTES-PE38 treatment group suggested that RANTES/CCL5 might not have had a role in the persistence or maintenance of these cells in the allergic airway.

### Example 12

RANTES-PE38 Treatment from Days 15 to 30 Significantly Increased Whole Lung Levels of MCP-1/CCL2 and Neutrophil Myeloperoxidase (MPO) at Day 30.

This example examined whole lung levels of MCP-1/CCL2 and neutrophil myeloperoxidase.

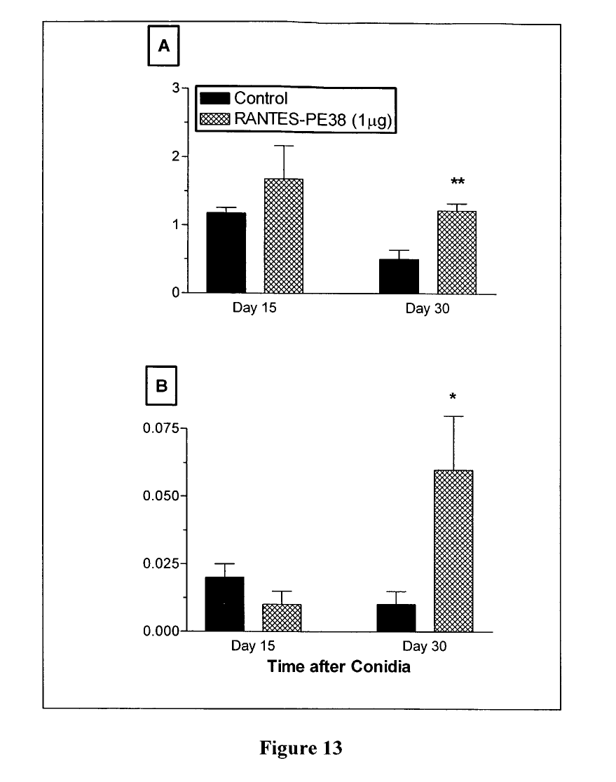

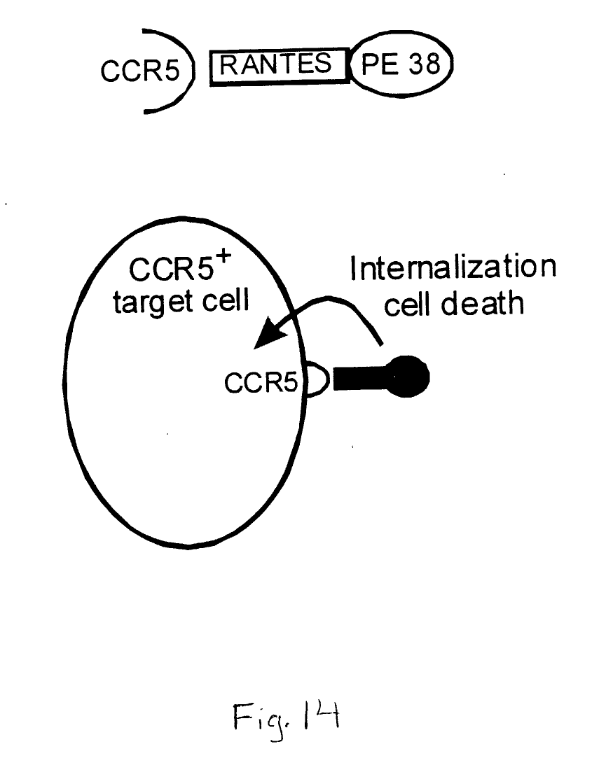

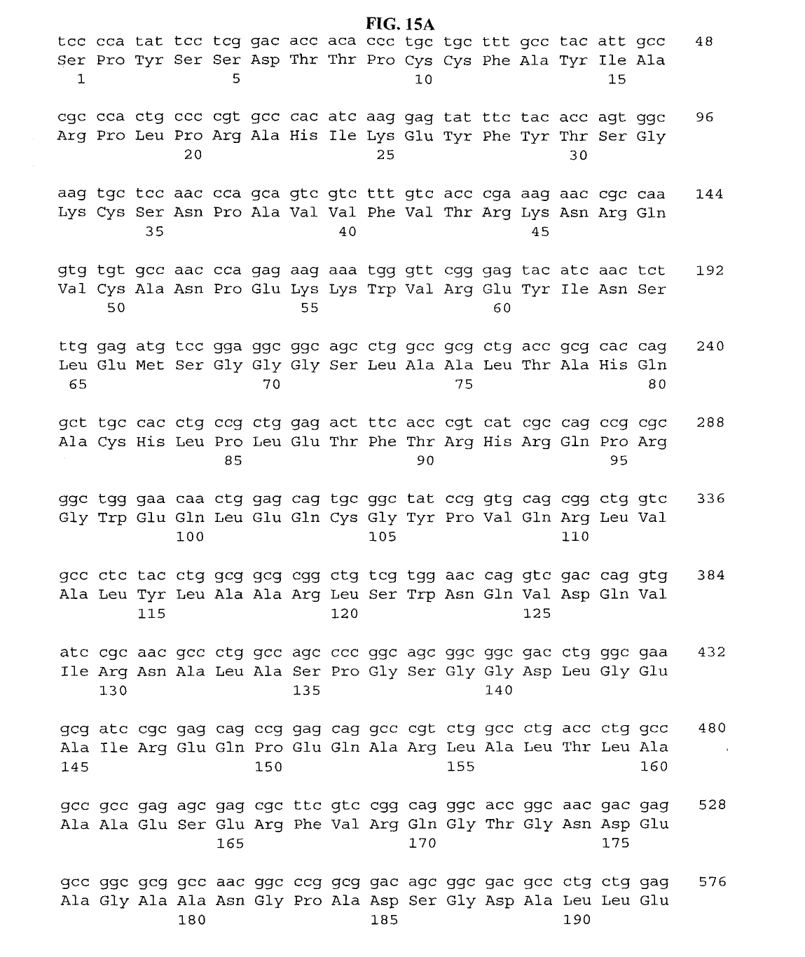

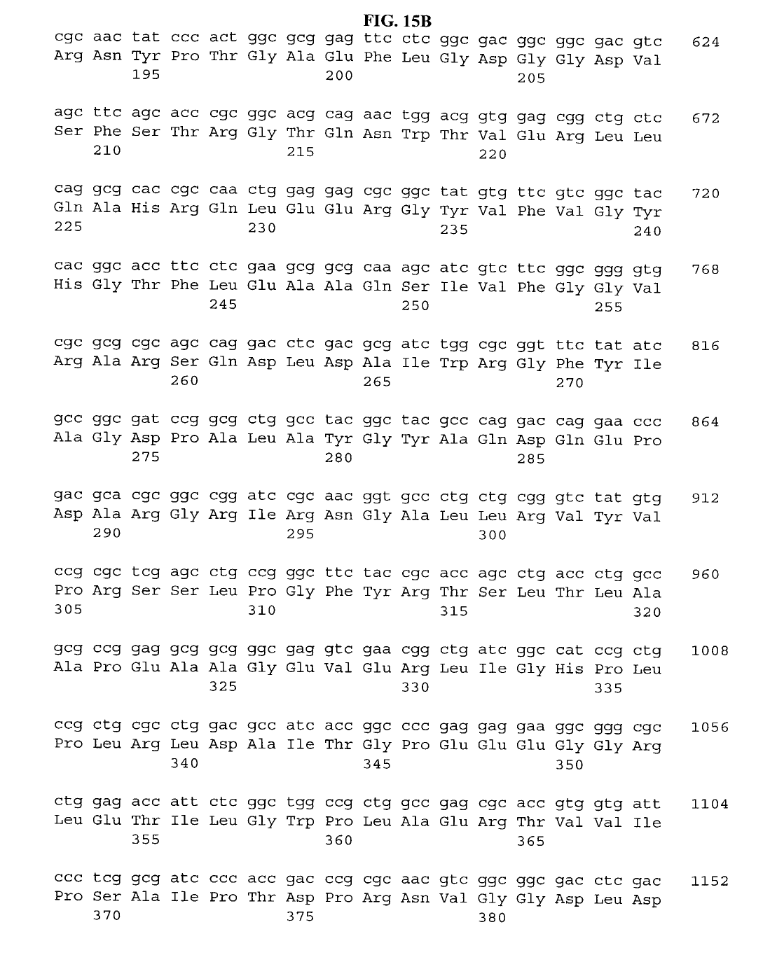

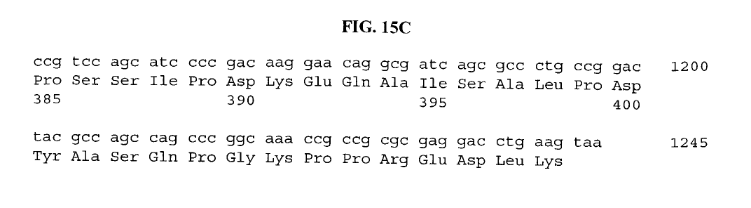

Further examination of whole lung samples from the control and RANTES-PE38-treated groups at day 30 after conidia suggested that targeting RANTES/CCL5-responsive cells from days 15 to 30 might have enhanced immune responses that sustained goblet cells in the airways despite the absence of other features of allergic airway disease. Recent evidence points to a major role for neutrophils in mucus hypersecretion associated with a number of allergic (39, 40) and non-allergic (41, 42) lung diseases. In addition, we have previously observed that the immunoneutralization of MCP-1/CCL2 from days 15 to 30 after conidia significantly attenuated the numbers of goblet cells in the airways of mice with A. fumigatus-induced allergic airway disease (43). With this background, it was investigated whether neutrophil activation and MCP1/CCL2 levels were altered by the RANTES-PE38 treatment from days 15 to 30 after conidia. As show in FIG. 13, whole lung levels of MPO (a specific marker of neutrophil activation) and MCP-1/CCL2 were significantly elevated in the RANTES-PE38-treated group at day 30 compared with the control group. Interestingly, the RANTES-PE38 treatment over the first 15 days of this model did not increase whole lung levels of MPO or MCP-1/CCL2, and goblet cell hyperplasia was not prominent in the airways of these mice. While not definitive, this data may explain why goblet cells in the airways of RANTES-PE38-treated mice at day 30 after conidia despite the profound reduction in peribronchial allergic inflammation and airway hyperreactivity in these mice at this time.

The most surprising finding pertaining to the targeting of RANTES/CCL5-responsive cells in the lungs of mice with chronic allergic airway disease was the marked reduction in the whole lung levels of CCR4 and CCR8 ligands, namely MDC/CCL22 and TARC/CCL17 (65, 66), and the reduced gene expression of both receptors after RANTES-PE38 treatment. Unlike most other CC chemokines, MDC/CCL22 and TARCC/CCL1 7 are encoded on human chromosome 16 (67) (68), and both chemokines appear to play a fundamental role in the function of dendritic cells, natural killer cells, monocytes, platelets and Th2 cells (29, 66, 69, 70). Previous studies in experimental models of allergic airway disease showed that both MDC/CCL22 (37), TARC/CCL17 (71) and CCR8 (64) were pivotal in the development of Th2-mediated responses including peribronchial eosinophilia and airway hyperreactivity. Recent clinical studies identify CCR4 and CCR8 as the key chemokine receptors expressed by airway T cells in allergen-challenged atopic asthmatics (72). In the present study, several observations indicate that the protective and therapeutic effects of RANTES-PE38 may have been mediated, in part, through its targeting or regulation of CCR4/CCR8 expressing cells in the allergic airways. As mentioned herein, the RANTES-PE38 treatment did not attenuate the recruitment of eosinophils and lymphocytes into the airways of allergic mice, data that contrasts with our previous observation that CCR5 gene deletion or anti-RANTES/CCL5 anti-serum treatment prevented the recruitment of both leukocyte cell types during allergic airway disease (29). Instead, the findings in RANTES-PE38-treated mice with chronic allergic airway disease model coincide more closely with observations regarding the role of MDC/CCL2 in an OVA allergic airway model. Specifically, MDC/CCL22 was shown to be involved in the transit and the peribronchial retention of leukocytes rather than in the regulation of leukocyte extravasation from the blood vessel or transepithelial migration into the airways (37). Further evidence that RANTES/CCL5 and MDC/CCL22 are linked during chronic allergic airway disease was obtained from the AdRANTES experiment in which the RANTES/CCL5 transgene significantly increased whole lung MDC/CCL22 levels.

The finding that RANTES-PE38 did not reverse or attenuate goblet cell hyperplasia typically present in A. fumigatus-sensitized mice at day 30 after conidia contrasts with our findings in mice lacking CCR5 (29) but matches our preliminary data regarding the progression of chronic allergic airway disease in mice that are lacking either CCR4 or CCR8. Briefly, both A. fumigatus-sensitized CCR4−/− and CCR8−/− mice exhibit significantly reduced airway inflammation and hyperreactivity at day 30 after conidia, but goblet cell hyperplasia is a prominent feature of airway remodeling in both chemokine receptor deficient mice (JMS and CMH unpublished findings). A profound neutrophil response during the course of allergic airway disease in CCR4−/− mice has also been observed. Neutrophil activation and MCP1/CCL2 levels were both significantly enhanced in whole lung samples from mice treated with RANTES-PE38 from days 15 to 30 after conidia. One or both of these mediators may have accounted for the sustained goblet cell hyperplasia in this model (39, 40, 43).

In conclusion, the present study showed that RANTES/CCL5 directly contributes to the development of chronic Aspergillus-induced allergic airway disease. The transgene-induced over-expression of RANTES/CCL5 markedly exacerbated existing disease, whereas the directed targeting of RANTES/CCL5-responsive cells with RANTES-PE38 resulted in the converse. RANTES-PE38 appeared to modulate chronic allergic airway disease through its direct targeting of CCR4/CCR8-expressing cells or RANTES/CCL5-responsive cells that regulate the recruitment of CCR4/CCR8-expressing cells into conidia-challenged lungs. Ongoing studies will address these two possibilities and in light of previous investigations, it is possible that this RANTES-PE38-susceptible cell is a T cell (72) or a dendritic cell (73). Overall, these data reveal the major role that RANTES/CCL5-responsive cells exert in the lung during Aspergillus-induced disease, and highlight the putative benefit that may be derived from targeting these cells during clinical allergy or atopic asthma.

### Example 13

Methods

Chronic Asperzillus-Induced Allergic Airway Disease in Mice.

Specific-pathogen free (SPF), female CBA/J mice (Jackson Laboratories; Bar Harbor, Me.) were housed in the University Laboratory Animal Medicine (ULAM) facility at the University of Michigan Medical School. Prior approval for mouse usage was obtained from ULAM. Mice were sensitized to a commercially available preparation of soluble A. fumigatus antigens as previously described in detail (31). A. fumigatus-sensitized mice received 5.0×106 A. fumigatus conidia suspended in 30 μl of 0.1% Tween-80 via an intratracheal inoculation (31).

Adenoviral-Mediated Overexpression of RANTES/CCL5 in the Lung.

A recombinant human type 5 adenovirus containing a murine RANTES/CCL5 cDNA (AdRANTES) was used to express biologically active RANTES/CCL5 in the lungs of A. fumigatus-sensitized mice during a conidia challenge. Previous use of this adenovirus construct revealed that the intratracheal administration of AdRANTES to rats caused the transient expression of RANTES/CCL5 in the bronchial epithelium for approximately 7 days (32). In the present study, A. fumigatus-sensitized mice received 2.0×10⁸ plaque forming units (PFU) of one of the recombinant viruses mixed with 5×10⁶ conidia at day 0. In the control group, 2.0×10⁸ PFU of adenovirus containing the LacZ gene (AdLacZ) was concomitantly administered with conidia. Lung RANTES/CCL5 levels, airway hyperresponsiveness, inflammation and remodeling were examined on days 3, 7, and 14 after the adenovirus and conidia challenge.

In Vivo Depletion of RANTES/CCL5-Responsive Cells in the Lung with a Chemokine Toxin.

A fusion protein of the chemokine RANTES/CCL5 and a truncated version of Pseudomonas exotoxin A (abbreviated RANTES-PE38) was used in the present study to target cells that express CCR5, a RANTES/CCL5 receptor (33). The construction of RANTES-PE38 is described in detail elsewhere (30). Previous studies have shown that RANTES-PE38 kills CCR5-positive Chinese hamster ovary cells but has no toxic effects towards CCR5-negative cells in vitro (30). Because RANTES-PE38 had not been previously used in vivo, we selected the intranasal route of administration (the same route employed in our previous studies with a chimeric protein composed of recombinant IL-13 and Pseudomonas exotoxin A (34, 35)) for this chimeric protein. Our initial study showed that a dose of 1000 ng, but not 200 ng, of RANTES-PE38 given intranasally had a significant inhibitory effect on the development of Aspergillus-induced allergic airway disease. The higher dose of RANTES-PE38 was subsequently used in the studies described below. Accordingly, groups of ten A. fumigatus-sensitized mice received RANTES-PE38 at the time of the conidia challenge and additional intranasal doses were given at two-day intervals up to day 15 after conidia. Other groups of five A. fumigatus-sensitized mice received an intranasal RANTES-PE38 at day 15 after conidia and additional intranasal doses were administered at two-day intervals up to day 30 after conidia. The control groups (n=5-10/group) received a similar quantity of normal saline alone according to the same protocols. Airway hyperresponsiveness, inflammation and remodeling were examined on either day 15 or 30 after the conidia challenge.

Measurement of Bronchial Hyperresponsiveness

Bronchial hyperresponsiveness in sodium pentobarbital (Butler Co., Columbus, Ohio; 0.04 mg/g of mouse body weight) anesthetized- and mechanically ventilated- (Harvard Apparatus, Reno, Nev.) mice was assessed using a Buxco™ plethysmograph (Buxco, Troy, N.Y.) (31). After a baseline period of 5 min, the mouse received doses of methacholine ranging from 420 to 625 μg/kg by tail vein injection. Airway hyperresponsiveness was calculated as previously described in detail (31). After the assessment of airway hyperresponsiveness, mice were euthanized and a bronchoalveolar lavage (BAL) was performed using 1 ml of normal saline, and a 500-μl aliquot of blood was also removed from each mouse. The BALs were centrifuged at 2000 rpm for 5 min, the supernatants were discarded and the pelleted cells were transferred to a Cytospin (Shandon Scientific, Runcorn, UK). Serum was obtained from each blood sample after centrifugation at 15000 rpm for 10 min. Finally, whole lungs were dissected from each mouse and snap frozen in liquid N₂ or prepared for histological analysis (see below).

Morphometric Analysis of Leukocyte Accumulation in BAL Samples

Neutrophils, macrophages, eosinophils, and lymphocytes were quantified in BAL samples cytospun onto microscope slides. Each slide was stained with a Wright-Giemsa differential stain, and the average number of each cell type was determined after counting a total of 300 cells in 15 high-powered fields (HPF; 1000×) per slide. A total of 1×10⁵ BAL cells were cytospun onto each slide to compensate for differences in cell retrieval.

RNA Isolation and GEArray Q Series Arrays

Total RNA was extracted from each whole lung sample using TRIzol reagent according to the manufacturer's directions (Invitrogen Life Technologies, Carlsbad, Calif.). The isolated RNA was quantified spectrophotometrically (260 nm), and equivalent amounts of RNA from control and RANTES-PE38-treated lungs were analyzed for chemokines and chemokine receptors using non-radioactive GEArray Q Series, (cDNA expression arrays), from SuperArray (Bethesda, Md.). Each array was exposed to photographic film for a standard period of time and the films are scanned to create Tiff files. The tiff files were analyzed using ScanAlyze software (courtesy of Michael Eisen, Stanford University) and the data from each array was expressed as integrated density values.

Chemokine ELISA Analysis

Murine RANTES/CCL5, eotaxin/CCL11, macrophage derived chemokine (MDC)/CCL22, TARC/CCL17 and monocyte chemoattractant protein-1 (MCP-1/CCL2) levels were determined in 50 μl aliquots of whole lung homogenates using a standardized sandwich ELISA technique (36). Nunc-immuno ELISA plates (MaxiSorp™) were coated with the appropriate polyclonal capture antibody (R&D Systems, Minneapolis, Minn.) at a dilution of 1-5 μg/ml of coating buffer (in M: 0.6 NaCl; 0.26 H₃BO₄; 0.08 NaOH; pH 9.6) overnight at 4° C. The unbound capture antibody was washed away and each plate was blocked with 2% BSA-PBS for 1 h at 37° C. Each ELISA plate was then washed three times with PBS tween 20 (0.05%; vol/vol), and 50 μl of undiluted or diluted (1:10) whole lung homogenate were added to duplicate wells and incubated for 1 h at 37° C. Following the incubation period, the ELISA plates were then thoroughly washed and the appropriate biotinylated polyclonal detection antibody (3.5 μg/ml) was added. After washing the plates 45-min later, streptavidin-peroxidase (Bio-Rad Laboratories, Richmond, Calif.) was added to each well for 30 min, and then thoroughly washed again. A chromagen substrate solution (Bio-Rad Laboratories) was added and optical readings at 492 nm were obtained using an ELISA plate scanner. Recombinant murine chemokines and cytokines were used to generate the standard curves from which the concentrations present in the samples were derived. The limit of ELISA detection for each cytokine was consistently above 50 pg/ml. Each ELISA was screened to ensure the specificity of each antibody used, and the cytokine and levels in each sample were normalized to total protein levels using the Bradford assay.

Measurement of Myeloperoxidase

Myeloperoxidase (MPO) is an enzyme found predominately in azurophilic granules of neutrophils that catalyzes the formation of hypochlorous acid. MPO is commonly used as an index of the activation state or presence of neutrophils in a variety of tissues. A commercially available MPO-specific ELISA was used to determine immunoreactive levels of MPO in BAL fluid per manufacturer's instructions.

Whole Lung Histological Analysis

Whole lungs from A. fumigatus-sensitized mice at various times after A. fumigatus conidia challenge were fully inflated with 4% paraformaldehyde, and dissected and placed in fresh paraformaldehyde for 24 h. Routine histological techniques were used to paraffin-embed the entire lung, and 5 μm sections of whole lung were stained with one of hematoxylin and eosin (H&E) and Periodic Acid Schiff (PAS). Inflammatory infiltrates and structural alterations were examined around blood vessels and airways using light microscopy at a magnification of 200×.

Statistical Analysis

All results are expressed as mean±standard error of the mean (SE). A Students t test was used to determine statistical significance between control and treatment groups at various times after the conidia challenge; P<0.05 was considered statistically significant.

TABLE I
CHEMOKINE RECEPTORS AND CHEMOKINE LIGANDS

| Chemokine | |
|---|---|
| Receptors | Chemokine Ligands |
| CXCR3 | 1-TAC (CXCL11), IP-10 (CXCL-10), Mig (CXCL9) |
| CXCR4 | SDF-1 (CXCL12) |
| CXCR5 | BCA1 (CXCL13) |
| CXCR6 | CXCL76 |
| CCR1 | MIP1 alpha (CCL3), RANTES (CCL5), MCP-3 (CCL7), |
| | MCP-4 (CCL13), HCC1 (CCL14), LKN1 (CCL15) |
| CCR2 | MCP-1 (CCL2), MCP-2 (CCL8), MCP-3 (CCL7), MCP-4 |
| | (CCL13) |
| CCR3 | RANTES (CCL5), MCP-2 (CCL8), MCP-3 (CCL7), |
| | MCP-4 (CCL13), eotaxin (CCL11), LKN1 (CCL15), |
| | MPIF-2 (CCL24), eotaxin-3 (CCL26) |
| CCR4 | TARC (CCL17), MDC (CCL22) |
| CCR5 | MIP1 alpha (CCL3), MIP1 beta (CCL4), RANTES (CCL5), |
| | MCP-2 (CCL8), MCP-3 (CCL7), MCP-4 (CCL13), eotaxin |
| | (CCL11) |
| CCR6 | LARC (CCL20) |
| CCR7 | ELC (CCL19), SLC (CCL21) |
| CCR8 | 1-309 (CCL1), MIP1 beta (CCL4), TARC (CCL17) |
| CCR9 | TECK (CCL25) |
| XCR1 | XCL1, XCL2 |
| CCR10 | CTACK (CCL27), MEC |

TABLE II
BAL LEUKOCYTE COUNTS IN CONTROL AND
RANTES-PE38-TREATED MICE AT DAYS 15 AND
30 AFTER CONIDIA CHALLENGE IN
A. FUMIGATUS-SENSITIZED MICE.

| | Day 15 | | Day 30 | |
|---|---|---|---|---|
| | Control | Day 15 | Control | Day 30 |
| Leukocyte | (Counts/ | RANTES-PE38 | (Counts/ | RANTES-PE38 |
| Type | HPF) | (Counts/HPF) | HPF) | (Counts/HPF) |
| Neutro- | 0 ± 0 | 0.1 ± 0.07 | 1.2 ± 0.5 | 0.4 ± 0.1 |
| phils | | | | |
| Eosino- | 1 ± 0.3 | 3 ± 0.4** | 0.2 ± 0.09 | 0.1 ± 0.07 |
| phils | | | | |
| Lympho- | 2.6 ± 0.5 | 3.2 ± 0.5 | 1 ± 0.2 | 0.2 ± 0.1# |
| cytes | | | | |
| Macro- | 11.3 ± 1.3 | 18 ± 1.4* | 16 ± 1.1 | 20 ± 1.6 |
| phages | | | | |

**P ≦ 0.01 compared with eosinophil counts in the control group at day 15 after conidia.
*P ≦ 0.05 compared with macrophage counts in the control group at day 15 after conidia.
#P ≦ 0.05 compared with lymphocyte counts in the control group at day 30 after conidia.

In the day 15 groups, mice received 1 μg of RANTES-PE38 in 20 μl of saline or 20 μl of saline alone by intranasal delivery approximately 2 h prior to the conidia challenge. Subsequent to this first treatment and the conidia challenge, each mouse received RANTES-PE38 or saline every 48-h for 15 days. In the day 30 groups, each mouse received 1 μg of RANTES-PE38 in 20 μl of saline or 20 μl of saline alone by intranasal delivery at day 15 after the conidia challenge. Subsequently, each mouse received RANTES-PE38 or saline every 48-h up to day 30 after conidia.

References

1. Schall et al. 1988. A human T cell-specific molecule is a member of a new gene family. J Immunol 141:1018-1025.
2. Schall et al. 1990. Selective attraction of monocytes and T lymphocytes of the memory phenotype by cytokine RANTES. Nature 347:669-671.
3. Alam et al. 1993. RANTES is a chemotactic and activating factor for human eosinophils. J Immunol 150:3442-3448.
4. Gonzalo et al. 1996. Eosinophil recruitment to the lung in a murine model of allergic inflammation. The role of T cells, chemokines, and adhesion receptors. J Clin Invest 98:2332-2345.
5. Folkard et al. 1997. Production of interleukin-8, RANTES and MCP-1 in intrinsic and extrinsic asthmatics. Eur Respir J 10:2097-2104.
6. Chihara et al. 1994. RANTES augments radical oxygen products from eosinophils. Int Arch Allergy Immunol 104 Suppl 1:52-53.
7. Chihara et al. 1997. Expression of mRNA for RANTES in human eosinophils. Int Arch Allergy Immunol 114 Suppl 1:33-35.
8. Hiura et al. 1999. Activation of the human RANTES gene promoter in a macrophage cell line by lipopolysaccharide is dependent on stress-activated protein kinases and the IkappaB kinase cascade: implications for exacerbation of allergic inflammation by environmental pollutants. Clin Immunol 90:287-301.
9. Pype et al. 1999. Expression of monocyte chemotactic protein (MCP)-1, MCP-2, and MCP-3 by human airway smooth-muscle cells. Modulation by corticosteroids and T-helper 2 cytokines. Am J Respir Cell Mol Biol 21:528-536.
10. Maune et al. 1997. Fibroblasts obtained from human nasal, laryngeal and tracheal mucosa produce the chemokine RANTES. Otolaryngol Pol 51:3-10.
11. Teran et al. 1999. Th1- and Th2-type cytokines regulate the expression and production of eotaxin and RANTES by human lung fibroblasts. Am J Respir Cell Mol Biol 20:777-786.
12. Propst et al. 2000. Proinflammatory and Th2-derived cytokines modulate CD40-mediated expression of inflammatory mediators in airway epithelia: implications for the role of epithelial CD40 in airway inflammation. J Immunol 165:2214-2221.
13. Berkman et al. 1996. Expression of RANTES mRNA and protein in airways of patients with mild asthma. Am J Respir Crit Care Med 154:1804-1811.
14. Stellato et al. 1995. Expression of the chemokine RANTES by a human bronchial epithelial cell line. Modulation by cytokines and glucocorticoids. J Immunol 155:410-418.
15. Wong et al. 1998. RANTES and MIP-1alpha activate stats in T cells. J Biol Chem 273:309-314.
16. Alam et al. 1996. Increased MCP-1, RANTES, and MIP-1alpha in bronchoalveolar lavage fluid of allergic asthmatic patients. Am J Respir Crit Care Med 153:1398-1404.
17. Powell et al. 1996. Increased expression of mRNA encoding RANTES and MCP-3 in the bronchial mucosa in atopic asthma. Eur Respir J 9:2454-2460.
18. Humbert et al. 1997. Bronchial mucosal expression of the genes encoding chemokines RANTES and MCP-3 in symptomatic atopic and nonatopic asthmatics: relationship to the eosinophil-active cytokines interleukin (IL)-5, granulocyte macrophage-colony-stimulating factor, and IL-3. Am J Respir Cell Mol Biol 16:1-8.
19. Holgate et al. 1997. Release of RANTES, MIP-1 alpha, and MCP-1 into asthmatic airways following endobronchial allergen challenge. Am J Respir Crit Care Med 156:1377-1383.
20. Tillie-Leblond et al. 2000. CC chemokines and interleukin-5 in bronchial lavage fluid from patients with status asthmaticus. Potential implication in eosinophil recruitment. Am J Respir Crit Care Med 162:586-592.
21. Tonnel et al. 2001. Characteristics of the Inflammatory Response in Bronchial Lavage Fluids from Patients with Status asthmaticus. Int Arch Allergy Immunol 124:267-271.
22. Venge et al. 1996. Identification of IL-5 and RANTES as the major eosinophil chemoattractants in the asthmatic lung. J Allergy Clin Immunol 97:1110-1115.
23. Lampinen et al. 1999. The role of interleukin-5, interleukin-8 and RANTES in the chemotactic attraction of eosinophils to the allergic lung. Clin Exp Allergy 29:314-322.
24. Kimata et al. 1996. RANTES and macrophage inflammatory protein 1 alpha selectively enhance immunoglobulin (IgE) and IgG4 production by human B cells. J Exp Med 183:2397-2402.
25. Fryer et al. 2000. The -403 G-->A promoter polymorphism in the RANTES gene is associated with atopy and asthma. Genes Immun 1:509-514.
26. Lukacs et al. 1996. C-C chemokine-induced cosinophil chemotaxis during allergic airway inflammation. J Leukoc Biol 60:573-578.
27. Lukacs et al. 1997. Differential recruitment of leukocyte populations and alteration of airway hyperreactivity by C-C family chemokines in allergic airway inflammation. J Immunol 158:4398-4404.
28. Gonzalo et al. 1998. The coordinated action of CC chemokines in the lung orchestrates allergic inflammation and airway hyperresponsiveness. J Exp Med 188:157-167.
29. Schuh et al. 2002. The role of CC chemokine receptor 5 (CCR5) and RANTES/CCL5 during chronic fungal asthma in mice. Faseb J 16:228-230.
30. Bruhl et al. 2001. Depletion of CCR5-expressing cells with bispecific antibodies and chemokine toxins: a new strategy in the treatment of chronic inflammatory diseases and HIV. J Immunol 166:2420-2426.
31. Hogaboam et al. 2000. Chronic airway hyperreactivity, goblet cell hyperplasia, and peribronchial fibrosis during allergic airway disease induced by Aspergillus fumigatus. Am. J. Pathol. 156:723-732.
32. Braciak et al. 1996. Overexpression of RANTES using a recombinant adenovirus vector induces the tissue-directed recruitment of monocytes to the lung. J Immunol 157:5076-5084.
33. Raport et al. 1996. Molecular cloning and functional characterization of a novel human CC chemokine receptor (CCR5) for RANTES, MIP-1beta, and MIP-1alpha. J Biol Chem 271:17161-17166.
34. Blease et al. 2001. IL-13 fusion cytotoxin ameliorates chronic fungal-induced allergic airway disease in mice. J Immunol 167:6583-6592.
35. Blease et al. 2002. Stat6-deficient mice develop airway hyperresponsiveness and peribronchial fibrosis during chronic fungal asthma. Am J Pathol 160:481-490.
36. Evanoff et al. 1992. A sensitive ELISA for the detection of human monocyte chemoattractant protein-1 (MCP-1). Immunol. Invest. 21:39-49.
37. Gonzalo et al. 1999. Mouse monocyte-derived chemokine is involved in airway hyperreactivity and lung inflammation. J Immunol 163:403-411.
38. Blease et al. 2000. Airway remodeling is absent in CCR1−/− mice during chronic fungal allergic airway disease. J Immunol 165:1564-1572.
39. Agusti et al. 1998. Goblet cell degranulation after antigen challenge in sensitized guinea pigs. Role of neutrophils. Am J Respir Crit Care Med 158:1253-1258.
40. Blyth et al. 1998. Induction, duration, and resolution of airway goblet cell hyperplasia in a murine model of atopic asthma: effect of concurrent infection with respiratory syncytial virus and response to dexamethasone. Am J Respir Cell Mol Biol 19:38-54.
41. Shimizu et al. 1996. Hypertrophic and metaplastic changes of goblet cells in rat nasal epithelium induced by endotoxin. Am J Respir Crit Care Med 153:1412-1418.
42. Nogami et al. 2000. Neutrophil elastase inhibitor, ONO-5046 suppresses ozone-induced airway mucus hypersecretion in guinea pigs. Eur J Pharmacol 390:197-202.
43. Blease et al. 2001. Antifungal and airway remodeling roles for murine monocyte chemoattractant protein-1/CCL2 during pulmonary exposure to Asperigillus fumigatus conidia. J Immunol 166:1832-1842.
44. Baggiolini and Dahinden. 1994. CC chemokines in allergic inflammation. Immunol Today 15:127-133.
45. Graziano et al. 1999. Cytokines, chemokines, RANTES, and eotaxin. Allergy Asthma Proc 20:141-146.
46. Conti et al. 1998. Will MCP-1 and RANTES take center stage in inflammatory diseases including asthma? Allergy Asthma Proc 19:121-123.
47. Zhang et al. 1994. RANTES: a novel mediator of allergic inflammation? Clin Exp Allergy 24:899-904.
48. Mule et al. 1996. RANTES secretion by gene-modified tumor cells results in loss of tumorigenicity in vivo: role of immune cell subpopulations. Hum Gene Ther 7:1545-1553.
49. Beck et al. 1997. Cutaneous injection of RANTES causes eosinophil recruitment: comparison of nonallergic and allergic human subjects. J Immunol 159:2962-2972.
50. Kuna et al. 1998. RANTES induces nasal mucosal inflammation rich in eosinophils, basophils, and lymphocytes in vivo. Am J Respir Crit Care Med 157:873-879.
51. Sato et al. 1999. Effects of reactive oxygen and nitrogen metabolites on RANTES- and IL-5-induced eosinophil chemotactic activity in vitro. Am J Pathol 155:591-598.
52. Boyce 1997. The pathobiology of eosinophilic inflammation. Allergy Asthma Proc 18:293-300.
53. Hogan et al. 1997. Aeroallergen-induced eosinophilic inflammation, lung damage, and airways hyperreactivity in mice can occur independently of IL-4 and allergen-specific immunoglobulins. J Clin Invest 99:1329-1339.
54. Umetsu and DeKruyff. 1997. Th1 and Th2 CD4+ cells in the pathogenesis of allergic diseases. Proc Soc Exp Biol Med 215:11-20.
55. McKenzie et al. 1998. A distinct role for interleukin-13 in Th2-cell-mediated immune responses. Curr Biol 8:339-342.
56. Akimoto et al. 1998. Abrogation of bronchial eosinophilic inflammation and airway hyperreactivity in signal transducers and activators of transcription (STAT)6-deficient mice. J Exp Med 187:1537-1542.
57. Kurup et al. 1994. IgE and cosinophil regulation in a murine model of allergic aspergillosis. J Leukoc Biol 56:593-598.
58. Hogaboam et al. 1999. Immunomodulatory role of C10 chemokine in a murine model of allergic bronchopulmonary aspergillosis. J Immunol 162:6071-6079.
59. Kurup et al. 1999. Aspergillus fumigatus antigen exposure results in pulmonary airway resistance in wild-type but not in IL-4 knockout mice. Clin Immunol 90:404-410.
60. Wells and Proudfoot. 1999. Chemokine receptors and their antagonists in allergic lung disease. Inflamm Res 48:353-362.
61. Owen 2001. Chemokine receptors in airway disease: which receptors to target? Pulm Pharmacol Ther 14:193-202.
62. Plater-Zyberk e al. 1997. Effect of a CC chemokine receptor antagonist on collagen induced arthritis in DBA/1 mice. Immunol Lett 57:117-120.
63. Teixeira et al. 1997. Chemokine-induced eosinophil recruitment. Evidence of a role for endogenous eotaxin in an in vivo allergy model in mouse skin. J Clin Invest 100:1657-1666.
64. Chensue et al. 2001. Aberrant in vivo T helper type 2 cell response and impaired eosinophil recruitment in CC chemokine receptor 8 knockout mice. J Exp Med 193:573-584.
65. Inngjerdingen et al. 2000. Human NK cells express CC chemokine receptors 4 and 8 and respond to thymus and activation-regulated chemokine, macrophage-derived chemokine, and I309. J Immunol 164:4048-4054.
66. Abi-Younes et al. 2001. The cc chemokines mdc and tarc induce platelet activation via ccr4. Thromb Res 101:279-289.
67. Nomiyama et al. 1998. Human chemokines fractalkine (SCYDI), MDC (SCYA22) and TARC (SCYA17) are clustered on chromosome 16q13. Cytogenet Cell Genet 81:10-11.
68. Imai et al.. 1998. Macrophage-derived chemokine is a functional ligand for the CC chemokine receptor 4. J Biol Chem 273:1764-1768.
69. Godiska et al. 1997. Human macrophage-derived chemokine (MDC), a novel chemoattractant for monocytes, monocyte-derived dendritic cells, and natural killer cells. J Exp Med 185:1595-1604.
70. Imai et al. 1999. Selective recruitment of CCR4-bearing Th2 cells toward antigen-presenting cells by the CC chemokines thymus and activation-regulated chemokine and macrophage-derived chemokine. Int Immunol 11:81-88.
71. Kawasaki et al. 2001. Intervention of thymus and activation-regulated chemokine attenuates the development of allergic airway inflammation and hyperresponsiveness in mice. J Immunol 166:2055-2062.
72. Panina-Bordignon et al. 2001. The C-C chemokine receptors CCR4 and CCR8 identify airway T cells of allergen-challenged atopic asthmatics. J Clin Invest 107:1357-1364.
73. Fischer et al. 2001. RANTES-induced chemokine cascade in dendritic cells. J Immunol 167:1637-1643.
74. Bertrand and Ponath. 2000. CCR3 Blockade as a New Therapy for Asthma. Exp. Opin. Invest. Drugs 9:43-52.

1. A method for treating a subject having allergic asthma, the method comprising:
(a) identifying a subject having allergic asthma, and
(b) administering to the subject a pharmaceutical composition comprising a chimeric polypeptide that comprises a first polypeptide domain comprising at least one moiety that specifically binds to a chemokine receptor; and, a second polypeptide domain comprising at least one of (a)-(d): (a) a moiety that binds to a T cell surface polypeptide, (b) a moiety that binds to a dendritic cell surface polypeptide, (c) a moiety that binds to a cell toxin, or (d) a cell toxin.

2. The method of claim 1, wherein the subject has or is at risk for chronic allergic asthma.

3. The method of claim 1, wherein the chemokine receptor is chemokine receptor 1 (CCR1), chemokine receptor 4 (CCR4) or chemokine receptor 5 (CCR5).

4. The method of claim 3, wherein the chemokine receptor is a human CCR1, CCR4 or CCR5.

5. The method of claim 3, wherein the moiety that specifically binds to the chemokine receptor comprises a RANTES or a fragment thereof.

6. The method of claim 3, wherein the moiety that specifically binds to the chemokine receptor comprises a MIP-1a or a fragment thereof.

7. The method of claim 1, wherein the second polypeptide domain comprises a cell toxin.

8. The method of claim 1, wherein the second polypeptide domain consists of a cell toxin.

9. The method of claim 1, wherein the cell toxin is cross-linked to the chimeric polypeptide.

10. The method of claim 5, wherein the second polypeptide domain comprises a cell toxin.

11. The method of claim 5, wherein the second polypeptide domain is a cell toxin.

12. The method of claim 1, wherein the chimeric polypeptide comprises a recombinant fusion protein.

13. The method of claim 1, wherein the moiety that specifically binds to the chemokine receptor comprises an antigen binding domain derived from an antibody that specifically binds to the chemokine receptor.

14. The method of claim 1, wherein the moiety that binds to a T cell surface polypeptide or a dendritic cell surface polypeptide comprises an antigen binding domain derived from an antibody that specifically binds to the T cell surface polypeptide or dendritic cell surface polypeptide.

15. The method of claim 14, wherein the T cell surface polypeptide is CD3.

16. The method of claim 1, wherein the dendritic cell surface polypeptide is an Eo1 chemokine receptor.

17. The method of claim 1, wherein the chimeric polypeptide comprises a first polypeptide domain that specifically binds to a chemokine receptor, and a second polypeptide domain that comprises a cell toxin.

18. The method of claim 17, wherein the chemokine receptor is CCR1, CCR4 or CCR5.

19. The method of claim 17, wherein the first polypeptide domain comprises a RANTES or a chemokine receptor binding fragment thereof.

20. The method of claim 1, wherein the cell toxin comprises a Pseudomonas exotoxin.

21. The method of claim 19, wherein the cell toxin comprises a Pseudomonas exotoxin.

22. The method of claim 21, wherein the Pseudomonas exotoxin comprises a PE38 exotoxin, a PE40 exotoxin or a PE37 exotoxin.

23. The method of claim 19, wherein the cell toxin comprises a diptheria toxin.

24. The method of claim 1, wherein the method further comprises the step of evaluating at least one symptom of allergic asthma in the subject.

25. The method of claim 24, wherein the symptom is IgE levels, goblet cell hyperplasia, peribronchial inflammation, recruitment of inflammatory leukocytes, airway hyperresponsiveness, coughing, wheezing, chest tightness, dyspnea, airway smooth muscle contraction, bronchial mucus secretion, inflammation, vasodilation, or release of inflammatory mediators.

26. The method of claim 1, further comprising the step of determining whether the administration of the chimeric polypeptide reduced the severity or initiation of one or more symptoms of allergic asthma in the subject.

27. The method of claim 1, wherein the chimeric polypeptide is administered once a day for a period of at least 7 days.

28. The method of claim 1, wherein the chimeric polypeptide is administered in a regimen of at least two cycles, wherein each cycle consists of: daily administration of the polypeptide for at least one day, followed by a rest period of at least one day in which the chimeric polypeptide is not administered.

29. The method of claim 1, wherein the chimeric polypeptide is administered at a unit dose of between 10 ng and 150 ug per kg of body weight of the subject.

30. The method of claim 1, wherein the subject's airway is hypersensitive to airborne fungus.

31. The method of claim 1, wherein the chimeric polypeptide is administered with a corticosteroid, bronchodilator, leukotriene antagonist, anti-inflammatory agent or therapeutic antibody.

32. The method of claim 19, wherein the chimeric polypeptide is administered in combination with a corticosteroid, bronchodilator, leukotriene antagonist, anti-inflammatory agent or therapeutic antibody.

33. The method of claim 1, wherein the chimeric polypeptide is formulated for intranasal, intratracheal, intrabronchial, intravenous or subcutaneous administration.

34. A method for treating a subject having allergic asthma, the method comprising:
(a) identifying a subject having allergic asthma, and
(b) administering to the subject a pharmaceutical composition comprising a nucleic acid encoding a chimeric polypeptide that comprises a first polypeptide domain comprising at least one moiety that specifically binds to a chemokine receptor; and, a second polypeptide domain comprising at least one of (a)-(d): (a) a moiety that binds to a T cell surface polypeptide, (b) a moiety that binds to a dendritic cell surface polypeptide, (c) a moiety that binds to a cell toxin, or (d) a cell toxin.

35. The method of claim 34, wherein the chimeric polypeptide comprises a RANTES or chemokine receptor binding fragment thereof.

36. The method of claim 34, wherein the second polypeptide domain comprises a cell toxin.

37. The method of claim 36, wherein the cell toxin comprises a Pseudomonas exotoxin.

38. The method of claim 37, wherein the Pseudomonas exotoxin comprises a PE38 exotoxin, a PE40 exotoxin or a PE37 exotoxin.

39. A method for treating a subject having allergic asthma, the method comprising:
(a) identifying a subject having allergic asthma, and
(b) administering to the subject a pharmaceutical composition comprising a chimeric polypeptide that comprises a first polypeptide domain comprising comprising a RANTES or a chemokine receptor binding fragment thereof, and a second polypeptide domain comprising a cell toxin.

40. The method of claim 39, wherein the cell toxin comprises a Pseudomonas exotoxin.

41. The method of claim 40, wherein the Pseudomonas exotoxin comprises a PE38 exotoxin, a PE40 exotoxin or a PE37 exotoxin.

42. The method of claim 39, wherein the cell toxin comprises a PE38 exotoxin.

43. The method of claim 39, wherein the pharmaceutical composition is administered in combination with an anti-asthma medication.

44. The method of claim 43, wherein the asthma medication is selected from the group consisting of: a corticosteroid, a bronchodilator, a leukotriene antagonist, an anti-inflammatory agent and a therapeutic antibody.

45. A kit for treatment of allergic asthma, comprising:
(1) a pharmaceutical composition comprising a chimeric polypeptide that comprises a first polypeptide domain comprising at least one moiety that specifically binds to a chemokine receptor; and, a second polypeptide domain comprising at least one of (a)-(d): (a) a moiety that binds to a T cell surface polypeptide, (b) a moiety that binds to a dendritic cell surface polypeptide, (c) a moiety that binds to a cell toxin, or (d) a cell toxin; and
(2) instructions to administer the pharmaceutical composition to treat allergen induced asthma in a subject.

46. The kit of claim 45, wherein the kit comprises specific instructions for administration of the pharmaceutical composition intranasally, intratracheally, intrabronchially, intravenously or subcutaneously.

47. A method of providing a treatment for allergen-induced asthma, the method comprising:
(a) providing a pharmaceutical composition comprising a chimeric polypeptide that comprises a first polypeptide domain comprising at least one moiety that specifically binds to a chemokine receptor; and, a second polypeptide domain comprising at least one of (a)-(d): (a) a moiety that binds to a T cell surface polypeptide, (b) a moiety that binds to a dendritic cell surface polypeptide, (c) a moiety that binds to a cell toxin, or (d) a cell toxin; and
(b) providing instructions to use the pharmaceutical composition to treat allergen-induced asthma.

48. The method of claim 47, wherein the instructions comprise instructions to administer the pharmaceutical composition intranasally, by inhalation, subcutaneously or intravenously.
